(19) United States
(12) Patent Application Publication
(10) Pub. No.: US 2024/0307309 A1
(43) Pub. Date: Sep. 19, 2024

(54) ZWITTERIONIC LIPID NANOPARTICLE COMPOSITIONS, AND METHODS OF USE

(71) Applicant: CORNELL UNIVERSITY, Ithaca, NY (US)

(72) Inventors: Sijin LUOZHONG, Ithaca, NY (US); Zhefan YUAN, Ithaca, NY (US); Shaoyi JIANG, Ithaca, NY (US); Xiaoran HU, Ithaca, NY (US); Sean BAILEY, Ithaca, NC (US)

(21) Appl. No.: 18/268,472

(22) PCT Filed: Dec. 21, 2021
(86) PCT No.: PCT/US2021/064639

§ 371 Date: Jun. 20, 2023

Related U.S. Application Data
(60) Provisional application No. 63/129,343, filed on Dec. 22, 2020.

(51) Int. Cl. A61K 9/14; A61K 31/7105

(52) U.S. Cl. CPC A61K 9/145; A61K 31/7105

(57) ABSTRACT

A lipid nanoparticle composition comprising: (i) at least one zwitterionic polymer-containing lipid in which a lipid moiety is covalently attached to a zwitterionic polymer; (ii) at least one non-cationic lipid selected from charged and uncharged lipids, but not attached to a polymer; (iii) at least one cationic or ionizable lipid; and (iv) at least one therapeutic substance, and optionally (v) cholesterol or derivative thereof. Also described herein are methods of delivering a therapeutic substance to a subject, the method comprising administering to the subject a lipid nanoparticle composition described above.

### CROSS REFERENCE TO RELATED APPLICATIONS

This application claims the benefit of priority from U.S. Provisional Application No. 63/129,343, filed on Dec. 22, 2020, which is herein incorporated by reference in its entirety.

### GOVERNMENT SUPPORT

This invention was made with government support under grant number 2002940 and 2103295 awarded by the National Science Foundation. The government has certain rights in the invention.

### FIELD OF THE INVENTION

The present invention generally relates to zwitterionic lipid nanoparticle (LNP) formulations for the encapsulation and delivery of therapeutic agents, such as nucleic acids. The present invention more specifically relates to the use of such LNP formulations, particularly non-PEGylated versions thereof, for delivery of therapeutic agents to treat a range of diseases or disorders, such as by gene therapy, or to vaccinate a subject.

### BACKGROUND OF THE INVENTION

In vivo delivery of nucleic acids, such as DNA, siRNA and mRNA, holds the potential to revolutionize vaccination, enzyme replacement therapies, and the treatment of genetic disorders. Compared to virus vectors, lipid nanoparticle delivery platforms are believed to have fewer safety issues. Since the first FDA approved LNP-siRNA drug in 2018, an increasing number of lipid nanoparticles have been developed and moved to clinical trials. LNPs typically consist of four components: ionizable cationic lipids, phospholipids, cholesterol, and polyethylene glycol (PEG)-lipids. Among them, PEG-lipids are commonly used to stabilize and protect the LNP structure.

Although PEG was initially presumed to be immunologically inert, their attachment to lipids or proteins is now known to introduce an additional cause of immune response due to haptenic effects, leading to anti-PEG antibodies (Abs). An increasing number of clinical reports highlight that anti-PEG Ab, instead of anti-protein Ab, is the main culprit for clinical problems in some cases. In the typical case of Pegloticase (Krystexxa), a PEGylated uricase product, more than 40% of refractory chronic gout patients receiving this drug suffered high-level anti-PEG Ab responses and consequently became non-responders to its treatment.

Anti-PEG Abs have impaired the efficacy of some PEG-conjugated proteins on the market. Besides induced anti-PEG antibodies, pre-existing anti-PEG Abs are critical to all PEGylated drugs. During the clinical trial in patients with acute coronary syndrome, severe allergic reactions occurred after a first dose of pegnivacogin, a pegylated RNA aptamer. Doxil®, PEGylated liposomal formulation for doxorubicin, is also reported to have immediate hypersensitivity reactions in some patients upon first injection. Thus, there is a yet unmet need to provide new lipid compositions that can replace the problematic components in conventional LNPs and provide improved functioning biopharmaceutical products.

### SUMMARY OF THE INVENTION

The present invention is foremost directed to novel lipid nanoparticle (LNP) compositions for delivering therapeutics, such as nucleic acids, to a subject. The LNP compositions described herein advantageously possess low immunogenicity, long circulation capabilities, and targeting ability. The LNP compositions can also be functionalized with a targeting agent to bind to specific cells or cellular components.

In a first aspect, the present disclosure is directed to LNP compositions containing at least the following components: (i) at least one zwitterionic polymer-containing lipid in which a lipid moiety is covalently attached to a zwitterionic polymer; (ii) at least one non-cationic lipid selected from charged and uncharged lipids not attached to a polymer; (iii) at least one cationic or ionizable lipid possessing a secondary, tertiary, or quaternary amino group; and (iv) at least one therapeutic substance. In a first set of embodiments, the lipid moiety in component (i) is a diacylglycerol (diacylglyceride). In a second set of embodiments, component (i) excludes a polyalkylene oxide (e.g., PEG) segment. In a third set of embodiments, the zwitterionic polymer in component (i) is a betaine polymer, or more particularly, a carboxy betaine polymer. In a fourth set of embodiments, the non-cationic lipid in component (ii) contains a zwitterionic moiety. In a fifth set of embodiments, component (ii) excludes a polyalkylene oxide segment. In a sixth set of embodiments, the non-cationic lipid in component (ii) is a phospholipid, such as phosphatidyl serine (PS) lipid. In a seventh set of embodiments, the cationic or ionizable lipid possesses a secondary, tertiary or quaternary group. In an eighth set of embodiments, the cationic or ionizable lipid excludes a polyalkylene oxide (e.g., PEG) segment. In a ninth set of embodiments, the lipid nanoparticle composition further comprises: (v) cholesterol or derivative thereof. In a tenth set of embodiments, the therapeutic substance is a nucleic acid molecule, such as an RNA, or more specifically, mRNA, or more specifically viral mRNA or more specifically linear or cyclic mRNA. Notably, any of the first through tenth embodiments may be combined to result in a LNP composition of the present invention.

In another aspect, the present disclosure is directed to a method of delivering a therapeutic substance to a subject by administering to the subject any of the lipid nanoparticle compositions described above, including any of the first through tenth embodiments described above. In a first set of embodiments, the lipid nanoparticle composition is delivered to cells of the subject. In a second set of embodiments, the therapeutic substance is a nucleic acid molecule, and administration thereof results in gene therapy of the subject. In a third set of embodiments, the therapeutic substance is a nucleic acid molecule, and administration thereof results in vaccination of the subject.

In another aspect, the present disclosure is directed to a lipid composition containing a lipid moiety attached to a secondary, tertiary, or quaternary amine group along with a functional group, which is negatively charged under physiological conditions. This lipid moiety can be

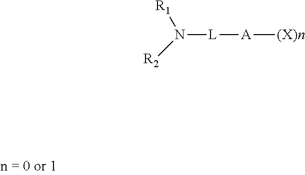

where
R1/R2=H or an alkyl group. The alkyl can be saturated or unsaturated, branched or unbranched, all-carbon and hydrogen or containing heteroatoms such as but not limited to N, O, F, Si, P, S, Cl, Br, and F.
L is a covalent linker group between N and A. The linker may be all carbon and hydrogen, or containing heteroatoms such as but not limited to N, O, F, Si, P, S, Cl, Br and F. The structure of L is exemplified by, but not limited to: —CH₂—, —CH₂CH(OH)—, —CH₂CHClCH₂—, —CH₂OCH₂—, —CH₂SCH₂—, —CH₂SSCH₂—, —CH₂COOCH₂—.
A-(X)n is a functional group that is negatively charged under physiological conditions. Structures are exemplified by, but not limited to the following:
   (i) A(X)n is a carboxylic acid group (A=-COOH and n=0) or
   (ii) A(X)n is a phosphate, where A=

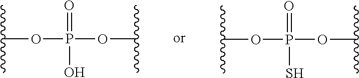 [SMILES: *OP(=O)(O)O* ; *OP(=O)(S)O*]

and n=1. X═H or an alkyl group that is saturated or unsaturated, branched or not branched, all-carbon or containing heteroatoms such as but not limited to N, O, F, Si, P, S, Cl, Br, and F or
   (iii) A(X)n is a sulfonic acid group, where A=

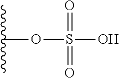 [SMILES: *OS(=O)(=O)O]

and n=0
(iv) A(X)n is a sulfonamide group, where A=

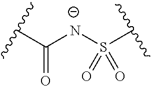 [SMILES: *C(=O)[N-]S(*)(=O)=O]

and n=1, X═H or an alkyl group that is saturated or unsaturated, branched or not branched, all carbon and hydrogen or containing heteroatoms such as but not limited to N, O, F, Si, P, S, Cl, Br, and F.

Exemplary structures of these ionizable lipids are

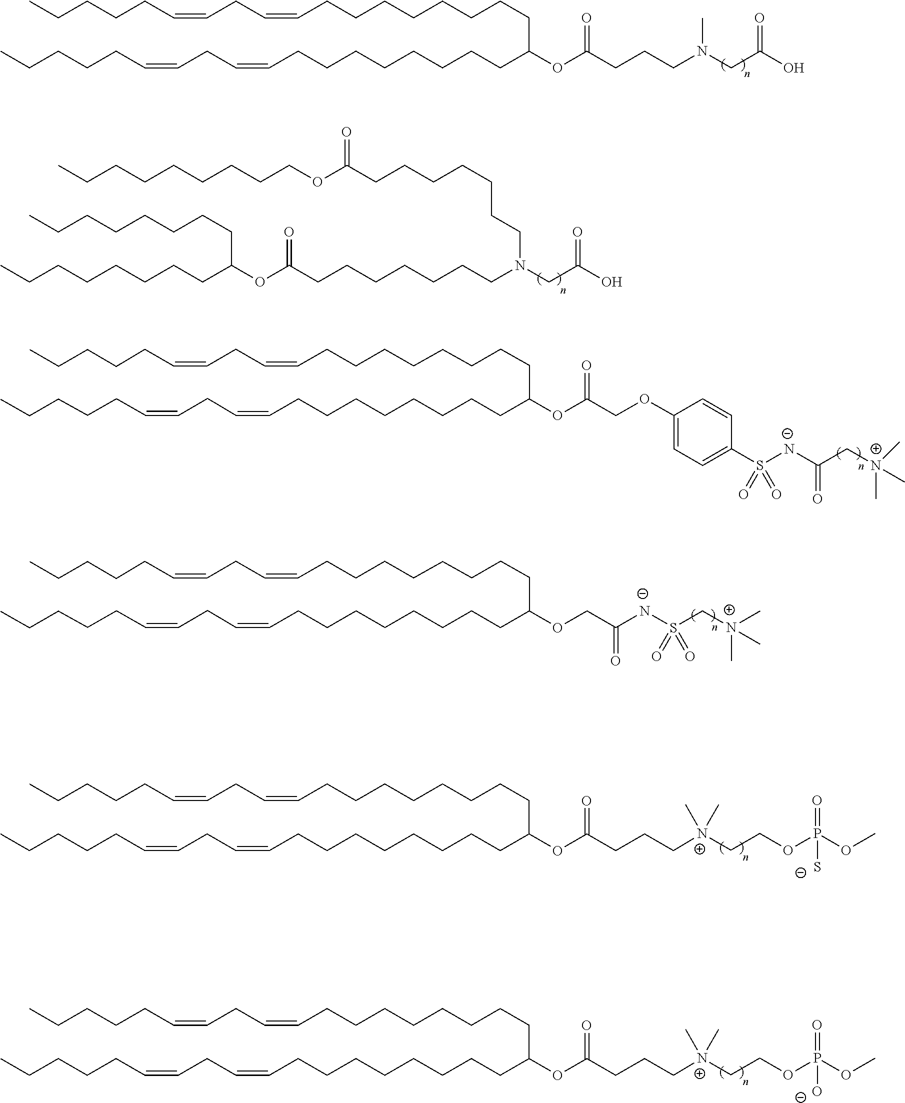

Where n=1 to 10

### BRIEF DESCRIPTION OF THE FIGURES

FIG. 1. The chemical structure and ¹H NMR spectrum of DMG-PCB (or PCB lipid) a zwitterionic polymer-containing lipid of the present invention.

FIG. 2. In vitro characterization of LNPs containing PCB lipid (or PCB-LNPs). (a) TEM image of a representative PCB-LNP formulation. (b) Luciferase expression of different PCB-LNPs in HepG2 cell line.

FIG. 3. In vivo mRNA expression of LNPs containing PCB lipid (or PCB-LNPs). Images were analyzed by Aura Image Software. All PCB-LNPs showed comparable transfection efficiency in livers to their PEG counterparts.

FIG. 4. Synthesis of ZW-A-CBn (n=1, 2, 3).

FIG. 5. ¹H NMR (CDCl3) spectrum of compound ZW-A-CB1.

FIG. 6. ¹H NMR (CDCl3) spectrum of compound ZW-A-CB2.

FIG. 7. ¹H NMR (CDCl3) spectrum of compound ZW-A-CB3.

FIG. 8. Synthesis of ZW-B-CBn (n=1, 2, 3).

FIG. 9. ¹H NMR spectrum of 3-((8-(nonyloxy)-8-oxooctyl)(8-(octadecan-9-yloxy)-8-oxooctyl)amino)propanoic acid (6).

FIG. 10. ¹H NMR spectrum of N-(8-(nonyloxy)-8-oxooctyl)-N-(8-(octadecan-9-yloxy)-8-oxooctyl)glycine (8).

FIG. 11. ¹H NMR spectrum of 4-((8-(nonyloxy)-8-oxooctyl)(8-(octadecan-9-yloxy)-8-oxooctyl)amino)butanoic acid (10).

FIG. 12. Synthesis of ZW-A-SulfAmid-3

FIG. 13. ¹H NMR (CDCl3) spectrum of compound ZW-A-SulfAmid-3

FIG. 14. A. Formulations of PCB-LNPs containing ZW-B-CB2 at different molar ratios. B. In vitro transfection was conducted in HepG2 cells for Group A and Group B. Each system was performed in three replicates.

FIG. 15. a) Injection scheme conducted in Example 8. b) Compositions of formulations studied in Example 8. c) EPO (erythropoietin) serum concentration analysis from sera drawn at indicated timepoint after the third injection of the formulation: CB1(left), CB2(middle), and MC3(right). Normalized data is shown here to represent the fold of change between two cohorts.

FIG. 16. In vivo luciferase expression of PCB-mLNP (i.e., PCB-LNP containing ZW-B-CB2 lipid) (left) and PS5-PCB-mLNP (i.e., PCB-LNP containing ZW-B-CB2 and PS lipids) (right) in C57B6/L mice (0.2 mg/kg). Images were taken 6 hours after intravenous injections.

FIG. 17. In vitro expression of Fluc-mRNA delivered by LNPs. pH value of the aqueous buffer solution: pH=5 (a) or pH=3 (b). Formulation: 50:38.5:10:1.5 (ionizable lipid+zwitterionic ionizable lipid): cholesterol: helper lipid (DOPE or DSPC): PEG-lipid. Horizontal stripes: MC3 50%+Zwitterionic lipid 0%; Vertical stripes: MC3 25%+ZW-B-CB1 25%; Checkerboard: MC3 25%+ZW-B-CB2 25%; Grid: MC3 25%+ZW-B-CB3 25%.

FIG. 18. In vitro expression of Fluc-mRNA delivered by LNPs. pH value of the aqueous buffer solution: pH=5 (left) or pH=3 (right). General formulation: 50:38.5:1.5 (ionizable lipid+zwitterionic ionizable lipid): cholesterol: PEG-lipid. Horizontal stripes: MC3 50%+Zwitterionic lipid 0%; Vertical stripes: MC3 25%+ZW-B-CB1 25%; Checkerboard: MC3 25%+ZW-B-CB2 25%; Grid: MC3 25%+ZW-B-CB3 25%.

FIG. 19. Luciferase expression of PS5-LNP and PSO-LNP (PEG formulations) in different cells: (a) HepG2, (b) Raw264.7, (c) primary mouse splenocytes. Experiments were done in three replicates. (d) IVIS images and bioluminescence signal analysis of organs isolated from mice treated with PSO-LNP and PS5-LNP carrying Fluc-encoding mRNA, respectively. The organs shown in the figure (from top to bottom) are lung, superficial cervical lymph nodes (SCLN, attached on saliva glands), liver, kidneys, and spleen.

FIG. 20. Structures of some exemplary zwitterionic polymer-containing lipids and non-cationic lipids with or without PC moiety in LNP formulations.

FIG. 21. Structures of some exemplary lipid compositions in which different types of lipid moieties (e.g., zwitterionic polymer modified lipids, cationic lipids, non-cationic lipids, and cholesterol or its derivative) are chemically combined.

### DETAILED DESCRIPTION OF THE INVENTION

In one aspect, the present disclosure is directed to a lipid nanoparticle (LNP) composition. Some LNPs of the art are described in, for example, X. Hou et al., Nature Reviews Materials, 6, 1078-1094, 2021, the contents of which are herein incorporated by reference. The term “lipid nanoparticle” refers to nanoparticles constructed, at least in part, of lipid molecules. As further described below, the lipid molecules include one or more zwitterionic polymer-containing lipids described herein and one or more non-cationic lipids, cationic or ionizable lipids, and/or cholesterol.

In different embodiments, the LNP has a size of precisely, about, at least, or up to, for example, 10, 20, 30, 40, 50, 60, 70, 80, 90, 100, 125, 150, 175, 200, 225, or 250 microns, or a size within a range bounded by any two of the foregoing values.

A first component of the LNP is at least one zwitterionic polymer-containing lipid. In the zwitterionic polymer-containing lipid, a lipid moiety is covalently attached (i.e., linked) to a zwitterionic polymer. The zwitterionic polymers are generally derived from zwitterionic monomers, as well as monomers that can be converted to zwitterionic monomers, i.e., precursors of zwitterionic monomers. Zwitterionic monomers are electronically neutral monomers that include equal numbers of positive and negative charges (e.g., one of each). In some embodiments, the zwitterionic polymer contains a plurality of repeating units, each repeating unit comprising one positive and one negative charged moiety. The zwitterionic polymer typically contains at least or greater than 2, 5, or 10, and up to or less than 100, 200, 300, 400, 500, or 1000 units.

“As used herein, the term “zwitterionic polymer” refers to a polymer prepared by polymerizing a polymerizable zwitterionic monomer, which provides a zwitterionic polymer having 100 mole percent zwitterionic moieties (i.e., each repeating unit of the zwitterionic polymer is a zwitterionic moiety; or refers to a polymer prepared by copolymerizing a polymerizable zwitterionic monomer and a polymerizable comonomer, which provides a zwitterionic polymer having less than 100 mole percent zwitterionic moieties (e.g., when the polymerizable zwitterionic monomer and the polymerizable comonomer are present in equal proportions in the polymerization mixture, the product is a zwitterionic polymer having 50 mole percent zwitterionic moieties).

The term “zwitterionic polymer” also refers to a polymer having a substantially equal number of negative (anionic) charges and positive (cationic) charges that is prepared by copolymerizing a polymerizable negatively charged monomer and a polymerizable positively charged monomer, each present in substantially equal proportions in the polymerization mixture. The product of such a copolymerization is a zwitterionic polymer having 100 mole percent zwitterionic moieties, where each zwitterionic moiety is defined as a pair of repeating units: a repeating unit having a negative charge and a repeating unit having a positive charge. Such zwitterionic polymers are referred to as mixed charge copolymers. The term “zwitterionic polymer” also refers to a polymer prepared by copolymerizing a polymerizable negatively charged monomer, a polymerizable positively charged monomer, each present in substantially equal proportions in the polymerization mixture, and a polymerizable comonomer, which provides a zwitterionic polymer having less than 100 mole percent zwitterionic moieties (e.g., when the combination of polymerizable negatively charged monomer and polymerizable positively charged monomer and the polymerizable comonomer are present in equal proportions in the polymerization mixture (i.e., 50% combination of polymerizable negatively charged monomer and polymerizable positively charged monomer and 50% polymerizable comonomer), the product is a zwitterionic polymer having 50 mole percent zwitterionic moieties).”

The lipid moiety is constructed of a polyol portion (e.g., a diol, glycerol, phosphatidylglycerol, phosphatidylethanolamine, or phosphatidylserine) that has been esterified with one or two fatty acid molecules to result in a monoacyl or diacyl lipid, wherein the term “acyl” refers to a RC(═O) group in which R is a linear or branched hydrocarbon (fatty) chain containing at least eight and typically up to 30 carbon atoms, wherein the hydrocarbon chain may be saturated or contain one or more carbon-carbon double bonds. The lipid moiety may be, for example, a diacyldiol (e.g., diacylethyleneglycol), diacylglycerol (diacylglyceride), diacylphosphatidylglycerol, diacylphosphatidylethanolamine, or diacylphosphatidylserine moiety. The lipid may be any of the lipids described in any one of Examples 1-14 provided in this application. The lipid may also be an of the lipids described in WO2011/057227, which is herein incorporated by reference. The fatty acyl portion may be derived from any of the known fatty acids. Some examples of fatty acyl portions include oleoyl, palmitoyl, lauryl, myristoyl, stearoyl, linoleoyl, and arachidonyl. The zwitterionic polymer is attached to the lipid moiety, such as any of the lipid moieties described above, typically via a carbon on the polyol. The zwitterionic polymer may contain the zwitterionic groups in side chains or the backbone (or combination thereof) of the polymer.

In one embodiment, the polymer is a homopolymer prepared from zwitterionic monomers and has the formula:

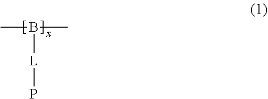

wherein B is a polymer backbone, such as a polyester, polyether, polyurethane, polyamide, or polyhydrocarbon (e.g., polyethylene or polypropylene) backbone, and P is a zwitterionic moiety. In particular embodiments, the backbone (B) may have any of the following structures:

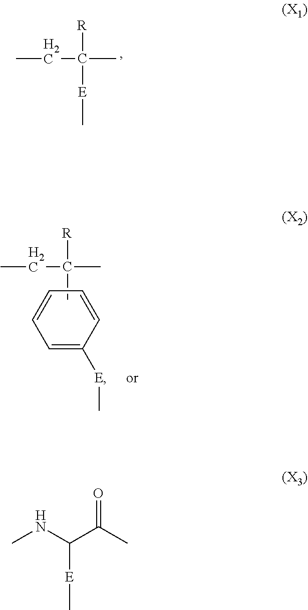

wherein R is selected from the group consisting of hydrogen and substituted or unsubstituted alkyl; and E is selected from the group consisting of substituted or unsubstituted alkylene, —(CH₂)ₚC(O)O—, and —(CH₂)ₚC(O)NR²—; p is typically an integer from 0 to 12; R² is selected from hydrogen and substituted or unsubstituted alkyl; and L is a straight or branched alkylene group optionally including one or more oxygen atoms. The subscript x is typically at least or greater than 2, 5, or 10, and up to or less than 100, 200, 300, 400, 500 or 1000 units.

In particular embodiments, P is selected from any of the following structures:

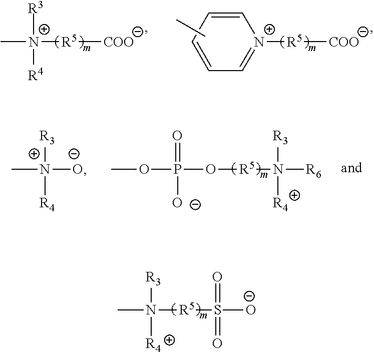

wherein R³, R⁴, and R⁶ are independently selected from the group consisting of hydrogen and substituted or unsubstituted alkyl group, R⁵ or R₅ is selected from the group consisting of substituted or unsubstituted alkylene, phenylene, and polyether groups, and m is an integer from 1 to 7; and x is an integer from 2 to 500.

In some embodiments, the zwitterionic polymer is a betaine polymer. In other embodiments, the zwitterionic polymer is a poly(phosphatidylcholine) polymer, poly(trimethylamine N-oxide) polymer, poly(zwitterionic phosphatidylserine) polymer, or glutamic acid-lysine (EK)-containing polypeptide. In some embodiments, zwitterionic phosphatidyl serine comprises one neighboring positive charged moiety to balance the negative charge of the phosphoserine. In some embodiments, zwitterionic phosphatidyl serine comprises a compound as described in “De novo design of functional zwitterionic biomimetic material for immunomodulation” Science Advances, 29 May 2020, Vol. 6, Issue 22, (DOI: 10.1126/sciadv.aba0754) which is hereby incorporated by reference in its entirety.

Some examples of betaine polymers include poly(carboxybetaine), poly(sulfobetaine), and poly(phosphobetaine) polymers. Suitable poly(carboxybetaine)s can be prepared from one or more monomers selected from, for example, carboxybetaine acrylates, carboxybetaine acrylamides, carboxybetaine vinyl compounds, carboxybetaine epoxides, and mixtures thereof. In one embodiment, the monomer is carboxybetaine methacrylate. Representative monomers for making carboxybetaine polymers useful in the invention include carboxybetaine methacrylates, such as 2-carboxy-N,N-dimethyl-N-(2′-methacryloyloxyethyl) ethanaminium inner salt; carboxybetaine acrylates; carboxybetaine acrylamides; carboxybetaine vinyl compounds; carboxybetaine epoxides; and other carboxybetaine compounds with hydroxyl, isocyanates, amino, or carboxylic acid groups. In a particular embodiment, the polymer is a poly(carboxybetaine methacrylate) (poly(CBMA)).

The zwitterionic polymer can be prepared by any suitable polymerization method, such as atom transfer radical polymerization (ATRP), reversible addition fragmentation chain transfer (RAFT) polymerization, and free radical polymerization. Any suitable radical initiators for polymerizing such monomers including those well known in the art, may be used. In some embodiments, to prepare the zwitterionic polymer-containing lipid or other polymer-containing lipid, a zwitterionic or other monomer or precursor thereof is attached to a lipid, and the monomer is polymerized while attached to the lipid. Alternatively, an already produced polymer may be attached to a lipid by means well known in the art.

In another embodiment, the zwitterionic polymer is a homopolymer that has a positive charge in the polymer backbone and a pendant carboxylic acid group and has the formula:

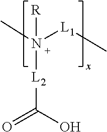

wherein R is selected from the group consisting of hydrogen and substituted or unsubstituted alkyl; L₁ and L₂ are independently a straight or branched alkylene group optionally including one or more oxygen atoms; and x is an integer from 2 to 500.

In another embodiment, the zwitterionic polymer is a mixed charge copolymer and has the general formula:

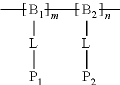

wherein B₁ and B₂ are independently selected from X₁, X₂, and X₃ as described earlier above; R is selected from hydrogen and substituted or unsubstituted alkyl; E is selected from substituted or unsubstituted alkylene, —(CH₂)ₚC(O)O—, and —(CH₂)ₚC(O)NR²—, wherein p is an integer from 0 to 12; R² is selected from hydrogen and substituted or unsubstituted alkyl; L is a straight or branched alkylene group optionally including one or more oxygen atoms; P₁ is a positively charged group; P₂ is a negatively charged group, such as a carboxylic acid group; m is an integer from 1 to 500; and n is an integer from 1 to 500. In some embodiments, P₁ is nitrogen in an aromatic ring or NR₅R₆, wherein R₅ and R₆ are independently substituted or unsubstituted alkyl group.

The positively charged unit (P₁ containing unit) of the zwitterionic polymer can be derived from a monomer having a positively charged pendant group. Representative monomers that can be used to derive the positively charged unit in the polymers of the present invention include 2-(dimethylamino)ethyl methacrylate, 2-(diethylamino)ethyl methacrylate, [2-(methacryloyloxy)ethyl] trimethylammonium chloride, and N-acetylglucosamine.

In one embodiment, the negatively charged unit of the zwitterionic polymer is derived from 2-carboxyethyl acrylate (CA), and the positively charged unit is derived from 2-(dimethylamino)ethyl methacrylate (DM). In another embodiment, the negatively charged unit is derived from 2-carboxyethyl acrylate (CA), and the positively charged unit is derived from 2-(diethylamino)ethyl methacrylate (DE). In another embodiment, the negatively charged unit is derived from 2-carboxyethyl acrylate (CA), and the positively charged unit is derived from [2-(methacryloyloxy)ethyl]trimethylammonium chloride (TM). In another embodiment, the negatively charged unit is derived from 2-carboxyethyl acrylate (CA), and the positively charged unit is derived from 2-aminoethyl methacrylate hydrochloride (NH₂).

In some embodiments, the zwitterionic polymer excludes a polyalkylene oxide (polyalkylene glycol) segment, or the zwitterionic polymer more specifically excludes a polyethylene oxide or polypropylene oxide segment. In some embodiments, all of component (i) excludes a polyalkylene oxide segment, or component (i) more specifically excludes a polyethylene oxide or polypropylene oxide segment. In some embodiments, the lipid nanoparticle as a whole excludes a polyalkylene oxide segment or molecule.

The term “lipid” refers to organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids. In one embodiment, lipid includes diacylglyceride.

In one embodiment, the zwitterionic polymer is linked with “compound lipids”. Some examples of such lipids include dioleoylphosphatidylglycerol (DOPG), dipalmitoylphosphatidylglycerol (DPPG), dioleoylphosphatidylethanolamine (DOPE), palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl-phosphoethanolamine, 16-O-dimethyl-phosphoethanolamine, 18-1-trans-phosphoethanolamine, 1-stearoyl-2-oleoyl-phosphatidyethanolamine (SOPE), and 1,2-dioleoyl-sn-glycero-3-phophoethanolamine (transDOPE). In another embodiment, the zwitterionic polymer is linked with “simple lipids”. In another embodiment, the zwitterionic polymer is linked with “derived lipids”. In some embodiments, the zwitterionic polymer comprises zwitterionic compounds as disclosed in WO2011057225A2, which is incorporated herein by reference.

A second component of the LNP is at least one non-cationic lipid selected from charged and uncharged lipids not attached to a polymer. The term “non-cationic lipid,” as used herein, refers to a lipid that is not positively charged and not capable of being ionized to a positively charged state. However, the non-cationic lipid may be neutral charged by containing a zwitterion (positive and negative charge within the polymer), such as any of the zwitterionic groups and moieties described earlier above

In some embodiments, the non-cationic lipid contains a zwitterionic moiety. The zwitterionic moiety can be any such moieties described in detail earlier above. The zwitterionic moiety may be, for example, a phosphobetaine, phosphatidylcholine, carboxybetaine, sulfobetaine, trimethylamine N-oxide, glutamic acid-lysine (EK)-containing, or zwitterionic phosphatidyl serine (phosphoserine) moiety, or a combination thereof. In some embodiments, the zwitterionic lipid is a phospholipid, such as a phosphatidylcholine or phosphatidylserine lipid.

In other embodiments, the non-cationic lipid is not zwitterionic but negatively charged by containing a negatively charged group (e.g., phosphoserine). A metal (e.g., alkali) or ammonium counteranion may be ionically and fluxionally associated with the negatively charged group.

In another set of embodiments, the non-cationic lipid is uncharged by not containing any charged groups. The uncharged non-cationic lipid may be, for example, a phosphatidylglycerol lipid, phosphatidylethanolamine lipid, or sphingolipid. The non-cationic lipid may, in some embodiments, be a simple lipid, such as a fat, oil, and/or wax. The non-cationic lipid may, in some embodiments, be a compound lipid, such as a phospholipid or glycolipid. The non-cationic lipid may, in some embodiments, be a derived lipid, such as a steroid, a phospholipid, a sphingolipid, and/or a sterol. In some embodiments, the non-cationic lipid is selected from a diacylphosphatidylethanolamine, a ceramide, a sphingomyelin, a dihydrosphingomyelin, a cephalin, or a cerebroside. In particular embodiments, the non-cationic lipid is selected from one or more of a phosphatidylethanolamine (PE), a phosphatidylglycerol (PG), a phosphatidic acid (PA), or a phosphatidylinositol (PI). In other particular embodiments, the non-cationic lipid is a dioleoylphosphatidylglycerol (DOPG), dipalmitoylphosphatidylglycerol (DPPG), dioleoylphosphatidylethanolamine (DOPE), palmitoyloleoyl-phosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl-phospho ethanolamine, 16-O-dimethyl-phosphoethanolamine, 18-1-trans-phosphoethanolamine, 1-stearoyl-2-oleoyl phosphatidyethanolamine (SOPE), and 1,2-dioleoyl-sn glycero-3-phophoethanolamine (transDOPE). Since the non-cationic lipid is not attached to a polymer, the non-cationic lipid excludes a polyalkylene oxide (polyalkylene glycol) segment.

In particular embodiments, non-cationic lipids containing an ionic moiety are phospholipids. In one embodiment, the non-cationic lipid containing an ionic moiety is a lipid conjugated with one or more carboxybetaine groups. In one embodiment, the non-cationic lipid containing an ionic moiety is a lipid conjugated with one or more sulfobetaine groups. In one embodiment, the non-cationic lipid containing an ionic moiety is a lipid conjugated with one or more trimethylamine N-oxide groups.

A third component of the LNP is at least one cationic or ionizable lipid. The cationic or ionizable lipid may or may not contain a lipid attached to a polymer that has a cationic group. In some embodiments, the cationic or ionizable lipid is not attached to a polymer. The term “cationic lipid,” as used herein, refers to a positively charged lipid (typically, by possessing an ammonium group). In the cationic lipid, the positively charged group is not associated with a negative charge within the cationic lipid. Thus, the cationic lipid is not a zwitterionic lipid. The term “ionizable lipid,” as used herein, refers to lipids that contain one or more groups capable of being ionized to result in a positive charge in the polymer. The ionizable lipid generally possesses a secondary, tertiary, or quaternary amino group, or particularly an alkylated amine, or more particularly, a monoalkylamine or dialkylamine group, any of which can be protonated or alkylated to result in an alkylated ammonium group. The cationic lipid may contain a trialkylamine group, which is necessarily positively charged when bound to the lipid. In particular embodiments, the cationic or ionizable lipid possesses a dimethylamino or trimethylamino (or dimethylammonium or trimethylammonium) group. In particular embodiments, the cationic or ionizable lipids are selected from 1,2-dioleoyl-3-dimethylammonium-propane (DODAP), 1,2-dilinoleyloxy-N,N-dimethyl-3-aminopropane (DLinDMA), 2,2-dilinoleyl-4-(2-dimethylaminoethyl)-[1,3]-dioxolane (DLinKC2DMA), and [(6Z,9Z,28Z,31Z)-heptatriaconta-6,9,28,31-tetraen-19-yl] 4-(dimethylamino)butanoate (DLinMC3DMA).

In some embodiments, the ionizable lipid contains a lipid moiety attached to a secondary, tertiary or quaternary amine group along with a functional group, which is negatively charged under physiological conditions. This lipid moiety can be

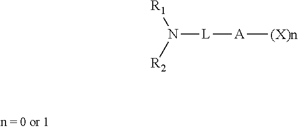

where
R1/R2=H or an alkyl group. The alkyl can be saturated or unsaturated, branched or unbranched, all-carbon and hydrogen or containing heteroatoms such as but not limited to N, O, F, Si, P, S, Cl, Br, and F.
L is a covalent linker group between N and A. The linker may be all carbon and hydrogen, or containing heteroatoms such as but not limited to N, O, F, Si, P, S, Cl, Br and F. The structure of L is exemplified by, but not limited to: —CH₂—, —CH₂CH(OH)—, —CH₂CHClCH₂—, —CH₂OCH₂—, —CH₂SCH₂—, —CH₂SSCH₂—, —CH₂COOCH₂—.
A-(X)n is a functional group that is negatively charged under certain pH conditions. Structures are exemplified by, but not limited to the following:
   (v) A(X)n is a carboxylic acid group (A=-COOH and n=O) or
   (vi) A(X)n is a phosphate, where A=

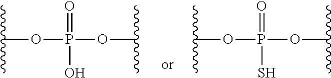 [SMILES: *OP(=O)(O)O* ; *OP(=O)(S)O*]

and n=1. X═H or an alkyl group that is saturated or unsaturated, branched or not branched, all-carbon or containing heteroatoms such as but not limited to N, O, F, Si, P, S, Cl, Br, and F or
   (vii) A(X)n is a sulfonic acid group, where A=

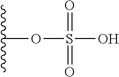 [SMILES: *OS(=O)(=O)O]

and n=0
(viii) A(X)n is a sulfonamide group, where A=

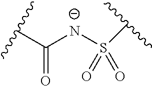 [SMILES: *C(=O)[N-]S(*)(=O)=O]

and n=1, X═H or an alkyl group that is saturated or unsaturated, branched or not branched, all carbon and hydrogen or containing heteroatoms such as but not limited to N, O, F, Si, P, S, Cl, Br, and F.

Exemplary structures of these ionizable lipids are

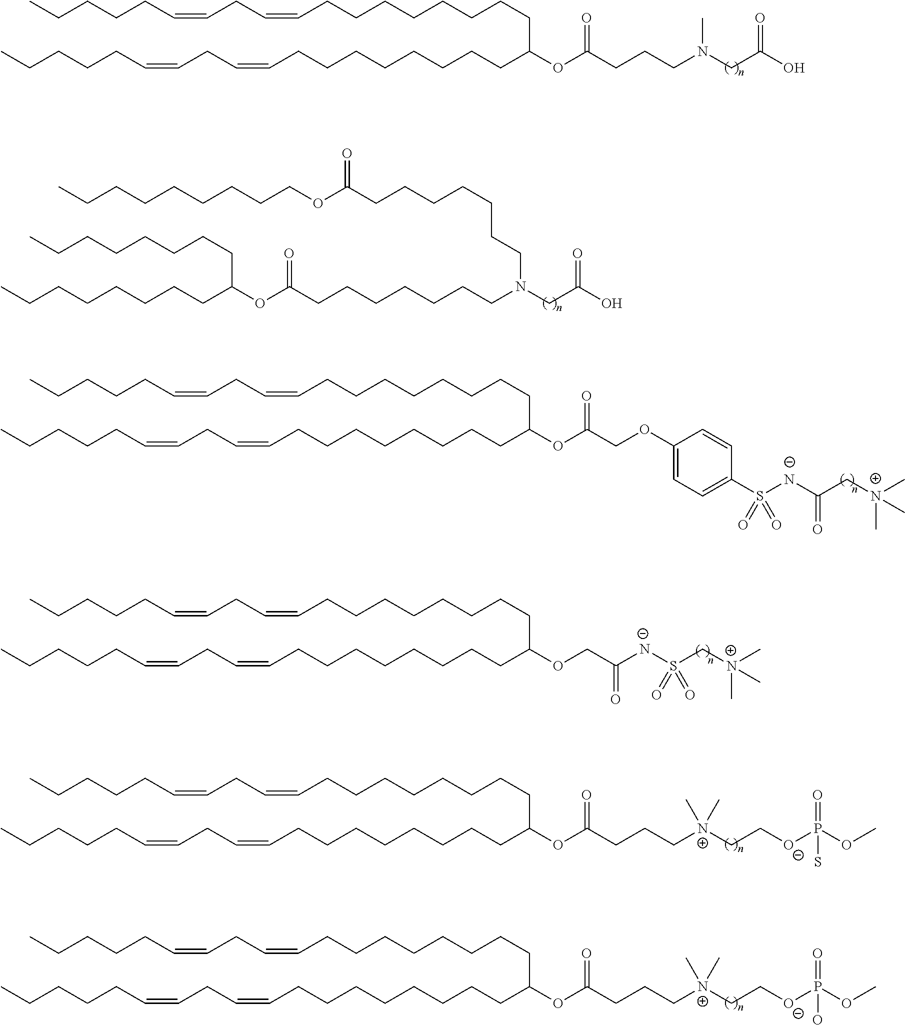

Where n=1 to 10

In some embodiments, the cationic or ionizable lipid excludes a polyalkylene oxide (polyalkylene glycol) segment, or the cationic or ionizable lipid more specifically excludes a polyethylene oxide or polypropylene oxide segment. In some embodiments, the cationic or ionizable lipid excludes a polyalkylene oxide segment, or the cationic or ionizable lipid more specifically excludes a polyethylene oxide or polypropylene oxide segment. In some embodiments, the lipid nanoparticle excludes a polyalkylene oxide segment.

In some embodiments, the LNP further includes cholesterol or a derivative thereof, which is considered herein to be an optional further component of the LNP. In certain embodiments, the cholesterol derivative is a phytosterol, e.g., β-sitosterol, campesterol, stigmasterol, fucosterol, or stigmastanol. In certain embodiments, the cholesterol derivative is dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N—(N′N′-dimethylaminoethyl)carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-30-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol, or fecosterol, or a salt or ester thereof, e.g., sodium cholate.

The LPN further includes a therapeutic substance incorporated into or encapsulated by the self-assembled shell constructed of one or more of the first, second, third, and/or fourth lipid components described above. The therapeutic substance can be any substance having therapeutic value for a living organism, particularly a mammal, such as a human or animal subject. The therapeutic substance may be, for example, a negatively charged nucleic molecule. The nucleic molecule may be, for example, a nucleotide, nucleoside, nucleobase, or a nucleic acid (e.g., DNA or RNA). In particular embodiments, the therapeutic substance contains one or more such nucleic molecules. In some embodiments, the therapeutic substance contains RNA, or more particularly, mRNA, or more particularly viral mRNA. In other particular embodiments, the therapeutic substance is a spike protein of a virus, such as a coronavirus, SARS-COV2 (COVID-19), or HIV virus.

The lipid nanoparticles and compositions of the present invention may be used for a variety of purposes, including the delivery of nucleic acid molecules, ribonucleoprotein (RNP) and numerous other therapeutic substances. Examples of nucleic acids include messenger RNA (mRNA), ribosomal RNA (rRNA), transfer RNA (tRNA), antisense oligonucleotide (ASO), short interfering RNAs (siRNA), microRNA (miRNA), miRNA inhibitors (antagomirs/antimirs), messenger-RNA-interfering complementary RNA (micRNA), multivalent RNA, circular RNA (circRNA), crispr RNA (crRNA), long noncoding RNA (lncRNA), plasmid DNA, oligo DNA, and complementary DNA (cDNA). In particular embodiments, the therapeutic molecule comprises one or more of DNA, RNA, ssDNA, dsDNA, ssRNA, dsRNA, and hybrids thereof. In other embodiments, the therapeutic molecule comprises one or more of plasmid DNA or linearized DNA. In other embodiments, the therapeutic molecule comprises one or more of messenger RNA (mRNA), small interfering RNA (siRNA), microRNA (miRNA), circular RNA (circRNA), and/or long-noncoding RNA (lncRNA). In other embodiments, the therapeutic molecule comprises antisense oligonucleotide (ASO). In other embodiments, the therapeutic molecule comprises Cas nuclease mRNA and/or guide RNA nucleic acid. The guide RNA nucleic acid may be, for example, a single-guide RNA (sgRNA). In any of the foregoing embodiments, the therapeutic molecule comprises a vaccine against SARS-Cov-2, particularly wherein the vaccine is an mRNA vaccine or wherein the mRNA vaccine corresponds to a spike protein or portion thereof.

In any of the foregoing embodiments, the nucleotide may encode fusion biological moieties comprising protective domains and functional domains. In some embodiments, the functional domains are fused to the protective domains directly or via a linker consisting of amino acids. In further particular embodiments, the protective domain may comprise: a plurality of negatively charged amino acids (e.g., aspartic acid, glutamic acid, and derivatives thereof); a plurality of positively charged amino acids (e.g., lysine, histidine, arginine, and derivatives thereof); and a plurality of additional amino acids independently selected from the group consisting of proline, serine, threonine, asparagine, glutamine, glycine, and derivatives thereof, wherein the ratio of the number of positively charged amino acids to the number of positively charged amino acids is from about 1:0.5 to about 1:2. In some embodiments, the protective domain can be selected from other amino acid polymers (e.g., extended recombinant polypeptide (XTEN), proline-alanine-serine and elastin-like polypeptides). In any of the foregoing embodiments, the protective domain can be selected from natural half-life extension domains (e.g., Fc fragment).

The LNP may also include lipid components with combined functionality. In embodiments, any of the lipid components, including but not limited to the zwitterionic polymer-containing lipid, the non-cationic lipid, the cationic lipid, and the cholesterol and/or cholesterol derivative, can include one or more functionalities of a different lipid component. In embodiments, the zwitterionic polymer-containing lipid includes the functionality of a zwitterionic polymer-containing lipid and a cationic or ionizable lipid. In embodiments, any of the zwitterionic polymer-containing lipid, the non-cationic or ionizable lipid, the cationic lipid, and the cholesterol and/or cholesterol derivative can also include one or more functionalities of one or more of the zwitterionic polymer-containing lipid, the non-cationic lipid, the cationic lipid, and the cholesterol and/or cholesterol derivative. In embodiments, a lipid component can include the functionality of another lipid component, thereby eliminating the need for an additional lipid component with that functionality. In embodiments, the zwitterionic polymer-containing lipid can include the functionality of the non-cationic lipid, thereby eliminating or reducing the need for the non-cationic lipid. In embodiments, the zwitterionic polymer-containing lipid can include the functionality of the cationic lipid, thereby eliminating or reducing the need for the cationic lipid. In embodiments, the zwitterionic polymer-containing lipid can include the functionality of the cholesterol and/or cholesterol derivative, thereby eliminating or reducing the need for the cholesterol and/or cholesterol derivative.

In embodiments, any lipid component (zwitterionic polymer modified lipids, cationic lipids, non-cationic lipids, and cholesterol or its derivative) may be chemically combined with any other lipid components. In embodiments, the zwitterionic polymer modified lipid and cationic lipid are chemically combined into one lipid. In embodiments, the zwitterionic polymer modified lipid and non-cationic lipid are chemically combined into one lipid. In embodiments, the cationic lipid and non-cationic lipid are chemically combined into one lipid. In embodiments, the zwitterionic polymer modified lipid, cationic lipid and non-cationic lipid are chemically combined into one lipid. In embodiments, the zwitterionic polymer modified lipid and cholesterol or a derivative are chemically combined into one lipid. In embodiments, the zwitterionic polymer modified lipid, cationic lipid and cholesterol or a derivative are chemically combined into one lipid. In embodiments, the zwitterionic polymer modified lipid, non-cationic lipid and cholesterol or a derivative are chemically combined into one lipid. In embodiments, the zwitterionic polymer modified lipid, non-cationic lipid, non-cationic lipids and cholesterol or a derivative are chemically combined into one lipid. In other embodiments, each lipid component is separate, distinct, and not combined with another lipid component.

Any of the components of the lipid nanoparticle described above may be chemically modified to contain a targeting ligand (e.g., Fab or Fc). In particular embodiments, the zwitterionic polymer-containing lipid possesses a targeting ligand to deliver LNPs loaded with a therapeutic or diagnostic agent or both of them to a targeted area within the special organ in the body, such as a peptide (e.g., RGD), a lipid (e.g., phosphoserine-containing lipid), a protein (e.g., apolipoprotein E), an aptamer (e.g., anti-VEGF aptamer), a sugar (e.g., Sialic acid) and an antibody (e.g., anti-PD1) or antibody fragment (e.g., Fab or Fc).

In another aspect, the present disclosure is directed to a method of delivering a therapeutic substance to a subject by administering to the subject any one or more of the LNP compositions described above. The subject is typically a mammal, more typically a human subject, but may also be another type of mammal, such as a pet or farm animal, such as a dog, cat, cow, or sheep. In some embodiments, the method of delivering the therapeutic substance results in a method of treating the subject. As a method of treatment, the LNP composition can be administered for the purpose of, for example, protein replacement therapy, cancer immunotherapy, cancer vaccine therapy, infectious disease vaccines, gene editing, autoimmune disease treatment, and/or cancer diagnosis. In particular embodiments, the LNP composition is administered for gene therapy comprising CRISPR-Cas gene editing, or for in vitro and in vivo production of extracellular vesicles, or for vaccination against coronavirus (e.g., SARS-CoV-2). In more particular embodiments, the LNP is used for clustered regularly interspaced short palindromic repeats-Cas endonuclease (CRISPR-Cas) gene editing in vitro and in vivo, for example including but not limited to delivering one or more nucleic acids that encode for one or more CRISPR associated proteins such as Cas protein. In some embodiments, the LNP is administered along with a checkpoint inhibitor (e.g., anti-Programmed death-ligand 1 (anti-PD-L1) antibody or anti-cytotoxic T-lymphocyte-associated protein 4 (anti-CTLA4) to treat cancer.

In some embodiments, the method of treatment includes targeted delivery of a therapeutic agent to a secondary lymphoid organ (SLO) in a subject, wherein the subject is administered lipid nanoparticles comprising a phosphoserine-containing lipid and the therapeutic agent. The SLO may be, for example, spleen and/or lymph nodes. The phosphoserine-containing lipid may be, for example, 1,2-dioleoyl-sn-glycero-3-phospho-L-serine (DOPS), or a naturally-occurring PS-lipid, such as L-α-phosphatidylserine (brain). In some embodiments, the targeted delivery results in cancer immunotherapy, autoimmune disease immunotherapy, or gene editing.

The LNP composition is typically administered in the form of a pharmaceutical composition containing the LNP. In the pharmaceutical composition, the LNP may be dissolved or suspended in, or admixed with, a pharmaceutically acceptable carrier, which may be a liquid, semi-solid (e.g., gel or wax), or solid, as well known in the art. The phrase “pharmaceutically acceptable” refers herein to those compounds, materials, compositions, and/or dosage forms which are, within the scope of sound medical judgment, suitable for administration to a subject. Each carrier should be “acceptable” in the sense of being compatible with the other ingredients of the formulation and physiologically safe to the subject. Any of the carriers known in the art can be suitable herein depending on the mode of administration.

Some examples of pharmaceutically acceptable liquid carriers include alcohols (e.g., ethanol), glycols (e.g., propylene glycol and polyethylene glycols), polyols (e.g., glycerol), oils (e.g., mineral oil or a plant oil), paraffins, and aprotic polar solvents acceptable for introduction into a mammal (e.g., dimethyl sulfoxide or N-methyl-2-pyrrolidone) any of which may or may not include an aqueous component (e.g., at least, above, up to, or less than 10, 20, 30, 40, or 50 vol % water). Some examples of pharmaceutically acceptable gels include long-chain polyalkylene glycols and copolymers thereof (e.g., poloxamers), cellulosic and alkyl cellulosic substances (as described in, for example, U.S. Pat. No. 6,432,415), and carbomers. The pharmaceutically acceptable wax may be or contain, for example, carnauba wax, white wax, bees wax, glycerol monostearate, glycerol oleate, and/or paraffins.

In some embodiments, the pharmaceutical composition contains solely the LNP and one or more solvents or the carrier. In other embodiments, the pharmaceutical composition includes one or more additional components. The additional component may be, for example, a pH buffering agent, mono- or poly-saccharide (e.g., lactose, glucose, sucrose, trehalose, lactose, or dextran), preservative, electrolyte, surfactant, or antimicrobial. If desired, a sweetening, flavoring, or coloring agent may be included. Other suitable excipients can be found in standard pharmaceutical texts, e.g. in “Remington's Pharmaceutical Sciences”, The Science and Practice of Pharmacy, 19th Ed. Mack Publishing Company, Easton, Pa., 1995.

The LNP composition, typically in the form of a pharmaceutical composition in which the LNP is admixed with or suspended in a liquid or solid pharmaceutically acceptable carrier, can be administered to the subject by any suitable route. The LNP may be administered intravenously, orally, intramuscularly, intradermally, subcutaneously, intranasally, or by inhalation. In particular embodiments, the LNP is administered by injection into the subject. In some embodiments, the LNP is delivered to cells of the subject. In some embodiments, the LNP is delivered by removing cells from the subject, administering the lipid nanoparticle to the removed cells, and then reintroducing the removed cells to the subject. In some embodiments, the LNP is injected directly in vivo and delivered into the host cells in vivo. In other embodiments, the LNP is transfected into the host cells ex vivo and the resulting cells are then infused in vivo.

Examples have been set forth below for the purpose of illustration and to describe the best mode of the invention at the present time. However, the scope of this invention is not to be in any way limited by the examples set forth herein.

### EXAMPLES

### Example 1. Preparation and Characteristics of a Zwitterionic Polymer Modified Lipid (DMG-PCB or PCB Lipid)

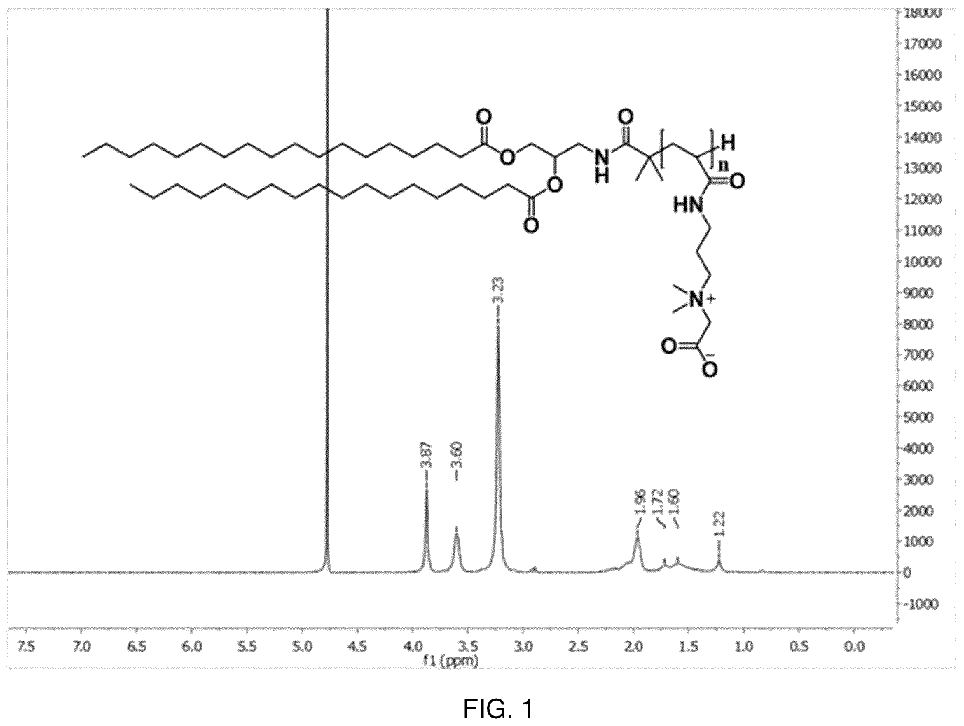

In this example, the preparation and characteristics of a representative zwitterionic polymer modified lipid of the invention, DMG-PCB are described here. The structure and ¹H NMR spectrum of the DMG-PCB are illustrated in FIG. 1.

DMG-N-BOC. Triethanolamine (1.6 mL, 9.36 mmol, 1.2 eqv.) was added to a solution of tert-Butyl N-(2,3-dihydroxypropyl) carbamate (1.5 g, 7.8 mmol, 1 eqv.) in dry DCM (10 mL) under a nitrogen atmosphere, and the mixture cooled in an ice bath to 0° C. To the solution, stearoyl chloride (2.83 g, 9.36 mmol, 1.2 eqv.) was dissolved in 20 mL DCM and added dropwise over 30 min. The reaction was stirred overnight at room temperature. The reaction mixture was extracted with saturated NaHCO₃ (aqueous [a.q.]). The organic layer was separated and washed with brine, then dried over MgSO4, filtered, and evaporated under vacuum. The residue was further purified by flash chromatography to obtain DMG-N-BOC (1.42 g, 40%).

DMG-N. DMG-N-BOC (100 mg) was dissolved in 5 mL of DCM and 5 mL of TFA. The reaction was stirred at room temperature for 3 hours. Then the solvent was removed in vacuo to obtain DMG-N as white powder.

CTA-DMG Lipid. A solution of DMG-N (100 mg, 0.16 mmol), 2-(Dodecylthiocarbonothioylthio)-2-methylpropionic acid N-hydroxysuccinimide ester (92.34 mg, 0.2 mmol) and TEA (22.3 μL, 0.16 mmol) in DCM (5 mL) was allowed to stir overnight at room temperature. The reaction mixture was concentrated under vacuum and purified by flash chromatography to obtain CTA-DMG lipid.

DMG-PCB. In a typical reversible addition-fragmentation chain-transfer (RAFT) polymerization reaction to yield a 10 kDa polymer, CTA-DMG lipid (100 mg, 0.142 mmol), and CBAAM-1-tBu (1036.32 mg, 2.84 mmol) 2,2′-azobis(2-methylpropionitrile) (AIBN, 4.66 mg, 0.028 mmol) were dissolved in anhydrous dimethylformamide (DMF, 5 mL) in a 25 mL round-bottom flask fitted with a stir bar. The flask was sealed with a rubber septum and purged with nitrogen for 30 min. The reaction was stirred overnight at 65° C. oil bath. The resultant polymer was precipitated in ethyl ester three times, centrifuged to collect pellet, and dried under vacuum overnight to yield a faintly yellow powder. To remove the trithiocarbonate group polymer (258.6 mg), the resultant polymer (258.6 mg, 0.05 mmol), EPHP (89.2 mg, 0.5 mmol) and AIBN (3.3 mg, 0.02 mmol) were dissolved in 3 mL of DMF. The solution was purged in nitrogen for 30 min and stirred at 100° C. for 2 hours. The product was precipitated in diethyl ether, centrifuged, and dried under vacuum to obtain white solid powder. The final product DMG-PCB was obtained by deprotecting the tert-Butyl groups with trifluoroacetic acid (TFA, 5 mL per 100 mg polymer) for 4 hours at room temperature, followed by precipitation in ethyl ether and centrifugation for three times. The pellet was dried overnight under vacuum, dissolved in RNase-free water, and dialyzed for two days. Molecular weight (around 12 kDa) was determined from ¹H NMR (D2O).

### Example 2. Preparation and In Vitro Characteristics of LNPs Containing Zwitterionic Polymer Modified Lipid (DMG-PCB or PCB Lipid)

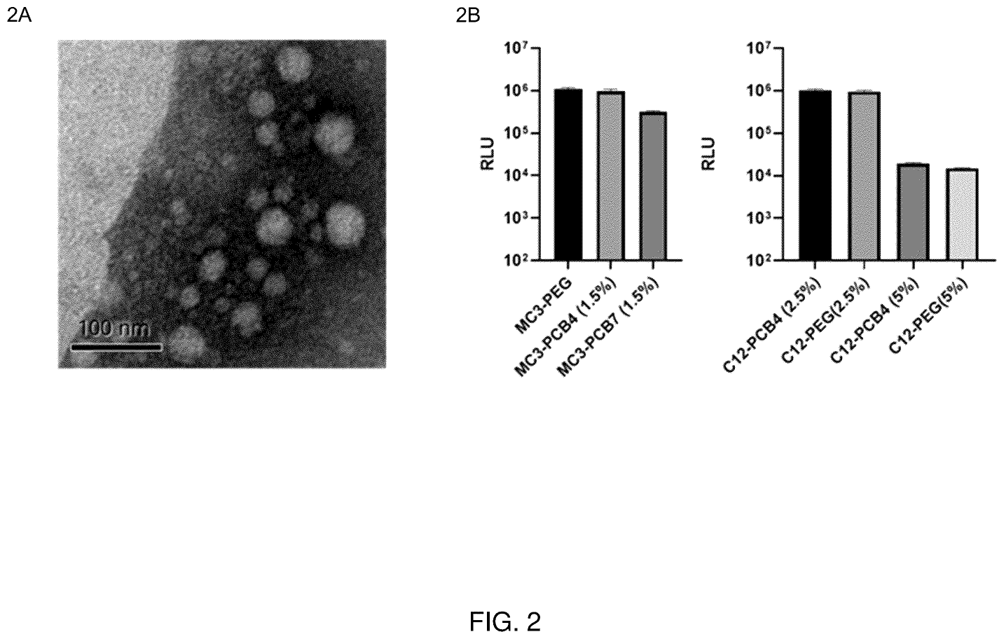

In this example, DMG-PEG from two commercially available LNP formulations is replaced by DMG-PCB from Example 1 to form LNPs containing PCB lipid (or PCB-LNPs). In vitro transfection efficiency of PCB-LNP is listed in FIG. 2b. The morphology of the PCB-LNP was observed under transmission electron microscopes (TEM) as shown in FIG. 2a. Preparation of PCB-LNP containing mRNA therapeutics is described below.

DLin-MC3-DMA (MC3) was purchased from Organixinc Inc. 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200) was purchased from Cordenpharma Inc. Other components include 1,2-distearoyl-sn-glycero-3-phosphocholine (DSPC, Avanti Polar Lipids, USA), cholesterol (Sigma, USA), and 1,2-dimyristoyl-rac-glycero-3-methoxypolyethylene glycol-2000 (DMG-PEG2K, Avanti Polar Lipids, USA). DMG-PCB containing different polymer molecular weight (4 kDa or 7 kDa) was synthesized from Example 1.

Encapsulation of mRNA into PCB-LNP was prepared by mixing lipid components and mRNA in a microfluidic mixing device NanoAssemblr Ignite (Precision NanoSystems Inc.). LNP formulations based on two different ionizable lipids were investigated respectively. For MC3-based LNPs, the molar ratio of the lipid mixture is MC3:DSPC:Cholesterol: X=50:40:38.5:1.5, where X is DMG-PEG2K for MC3-PEG, DMG-PCB4K for MC3-PCB4 and DMG-PCB7K for MC3-PCB7. For C12-200-based LNPs, the molar ratio of the lipid mixture is C12-200:DOPE:Cholesterol:Y=35:16:46.5:2.5, where Y is DMG-PEG2K for C12-PEG, DMG-PCB4K for C12-PCB. Briefly, four lipid components were dissolved in ethanol and separately, mRNA encoding firefly luciferase (Fluc) was dissolved in 50 mM citrate buffer (pH=3). Then, the aqueous phase and ethanol phase were mixed at a flowrate ratio of 3:1 in the microfluidic device. To remove residual organic solvents, the resulting LNP was washed with PBS in a 100-kDa centrifugal filter (MilliporeSigma, USA).

LNPs were transfected to HepG2 (ATCC No. HB-8065) and luciferase expression was analyzed at 6-h after transfection. Briefly, cells were plated at 60-70% confluency and incubated at 37° C. with 5% CO₂ in Eagle's Minimum Essential Medium (EMEM) supplemented with 10% fetal bovine serum (FBS) and 1× Penicillin-Streptomycin (Pen-Strep). During transfection, RNA was added per well at a dose indicated in FIG. 2b. After 6 h of transfection, the culture medium containing LNPs was carefully removed, and the cells were rinsed once with PBS gently. Luciferase expression was measured using a luciferase assay (Promega Cat #: E1501) following the manufacture's protocol. All PCB-LNPs showed in vitro transfection efficiency comparable to that of their corresponding PEG-LNPs, indicating that DMG-PCB can be used in the LNPs to delivery mRNA into the cells.

### Example 3. In Vivo Delivery of LNPs Containing Zwitterionic Polymer Modified Lipid (DMG-PCB or PCB Lipid)

PCB-LNP formulations from Example 2 were delivered to mice to study the biodistribution of protein expression after systemic delivery.

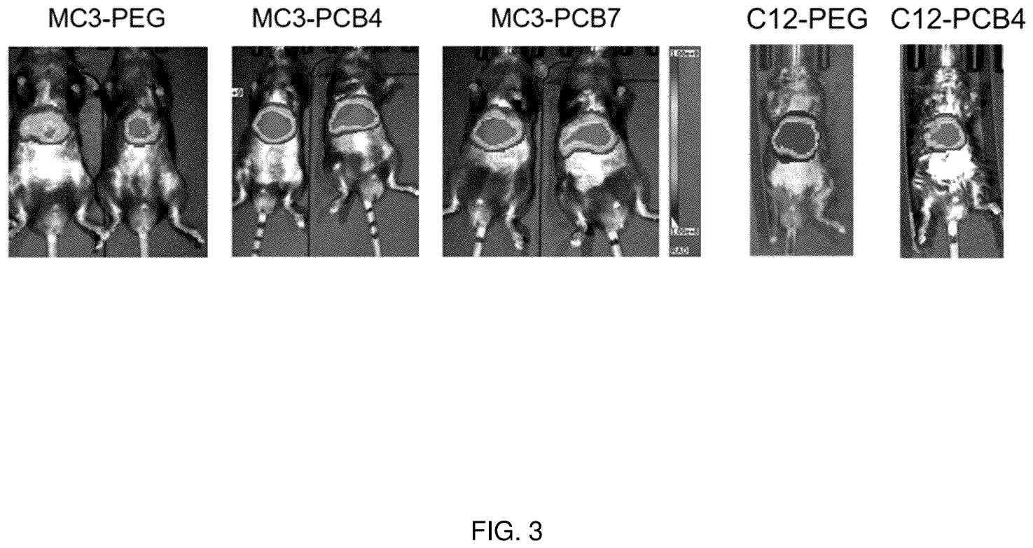

Briefly, C57BL/6 mice at the age of 6-8 weeks were administered with LNPs carrying Fluc-mRNA via retro-orbital injections at a dosage of 0.4 mg/kg. Bioluminescence imaging was performed at 6-h after transfection. Mice were injected with 150 mg/kg of D-luciferin diluted in PBS. All images were analyzed using Aura Image Software. As shown in FIG. 3, all PCB-LNPs facilitated high mRNA expression in liver. Efficient in vivo expression of mRNA was corroborated in two different LNP systems: one using MC3 as ionizable lipid; another one using C12-200 as ionizable lipids. Therefore, incorporation of this super-hydrophilic polymer conjugated lipid into LNPs performed efficient in vivo transfection, indicating DMG-PCB as a feasible alternative to DMG-PEG.

### Example 4. Synthesis and Characterization of ZW-A-CBn (n=1,2,3)

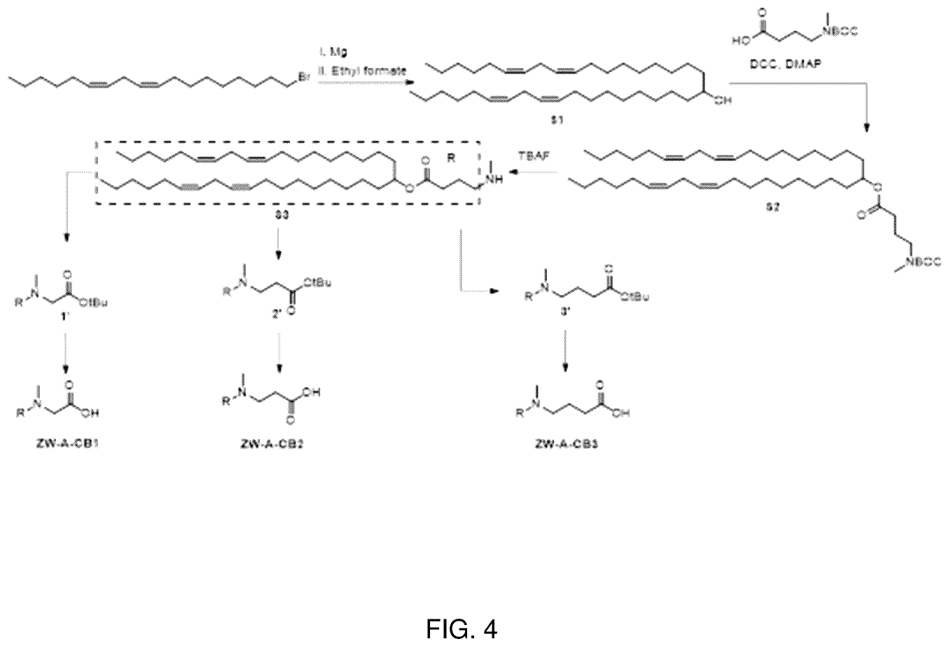

In this example, ZW-A-CBn (n=1,2,3) was synthesized and characterized by ¹H-NMR. The synthesis method of ZW-A-CBn is illustrated in FIG. 4 and described below.

Compound S1 was synthesized following Jayaraman and Hope's conditions (Angew. Chem. Int. Ed. 2012, 51, 8529-8533). Mg turnings were freshly activated with 0.1 M HCl, washed with water and acetone, and dried under high vacuum. Mg (680 mg) was added into a three neck round bottom flask equipped with a magnetic stirrer, an addition funnel, and a reflux condenser. The flask was degassed and filled with 2.3 mL anhydrous ether. Linoleyl bromide (7.50 g) was dissolved in 14 mL ether, then 2 mL of the ether solution was added to the flask at once at room temperature. The reaction mixture was stirred for 2 min, then a small crystal of iodine was added. The dark brown color from the iodine faded away rapidly, and the reaction mixture started refluxing. The rest of the linoleyl solution in ether was added dropwise over 5-10 min while maintaining the reaction under gentle reflux. After addition, the reaction mixture was kept in a 35° C. water bath for 3 h and then cooled in an ice bath. Ethyl formate (0.8 mL, 0.734 g) was added dropwise at 0° C., and the reaction was allowed to warm up to room temperature. After 6 h, NH₄Cl solution was added to quench the reaction. The solution was extracted by ether (×3), dried using Na₂SO₄, and concentrated to yield a crude mixture as a yellow oil. A major byproduct was identified to be the formate ester of S1, so the crude mixture was dissolved in 10 mL saturated solution of KOH in ethanol and stirred overnight to hydrolyze the byproduct. The reaction mixture was then diluted with DCM, washed with NH₄Cl, concentrated, dried using Na₂SO₄, and then purified via silica gel flash chromatography (1% to 15% ethyl acetate in hexanes) to yield the desirable product as a light yellow oil. Yield: 3.56 g.

Compound S2: To a two neck round bottom flask under nitrogen was added S1 (2.00 g), 4-[(tert-butoxycarbonyl)(methyl)amino]butanoic acid (0.87 g), DMAP (0.045 g), and 25 mL DCM. The solution was cooled to 0° C., then a 5 mL solution of DCC in DCM was added dropwise. The reaction was allowed to warm to room temperature and stirred for 10 h. The solution was filtered, concentrated, and purified via silica gel flash chromatography using 0-10% ethyl acetate in hexane. 2.658 g colorless oil was produced as the product.

Compound S3: To a 50 mL round bottom flask was added S2 (1.758 g) and 5 mL 50 v/v % solution of TFA in DCM. The reaction was stirred at room temperature for 1.5 h, then concentrated. The crude was dissolved in DCM, washed with Na₂CO₃ solution, dried with Na₂SO₄, and concentrated. The yellow oil was purified via silica gel flash chromatography (2-15% MeOH in DCM) to yield 1.02 g faint yellow oil.

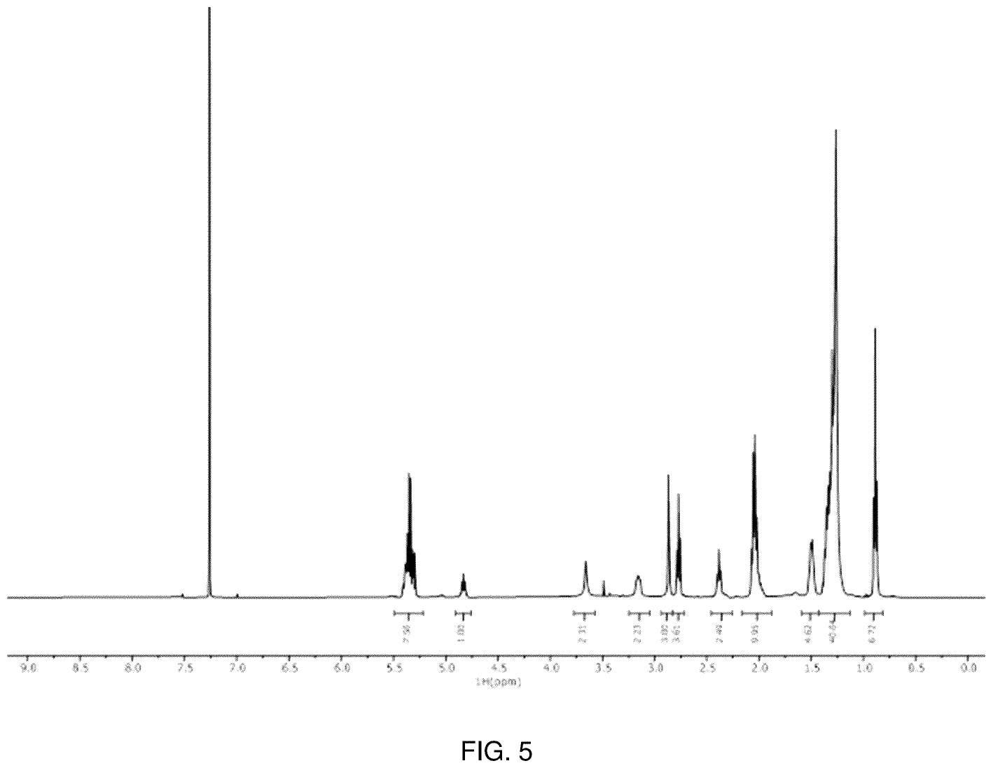

Compound ZW-A-CB1: To a 2 mL autosampler vial was added compound S3 (114 mg), tert-butyl bromoacetate (35.4 mg), K₂CO₃ (80 mg), KI (4.3 mg), and a mixture of anhydrous acetonitrile (0.3 mL) and THE (0.1 mL). The mixture was stirred at rt for 15 h, then diluted by DCM and filtered. The filtrate was concentrated and purified via silica gel flash column chromatography (5%-15% MeCN in DCM) to yield compound 1′ as a colorless oil. Yield: 61 mg. Compound 1′ (61 mg) was dissolved in a 50 v/v % solution of TFA in DCM and stirred at room temperature for 2 h. The reaction mixture was concentrated, then purified via silica gel flash chromatography using 5-20% MeOH in DCM to yield compound ZW-A-CB1 as a faint yellow oil. Yield: 53 mg. ¹H NMR (CDCl3) of compound ZW-A-CB1 is shown in FIG. 5

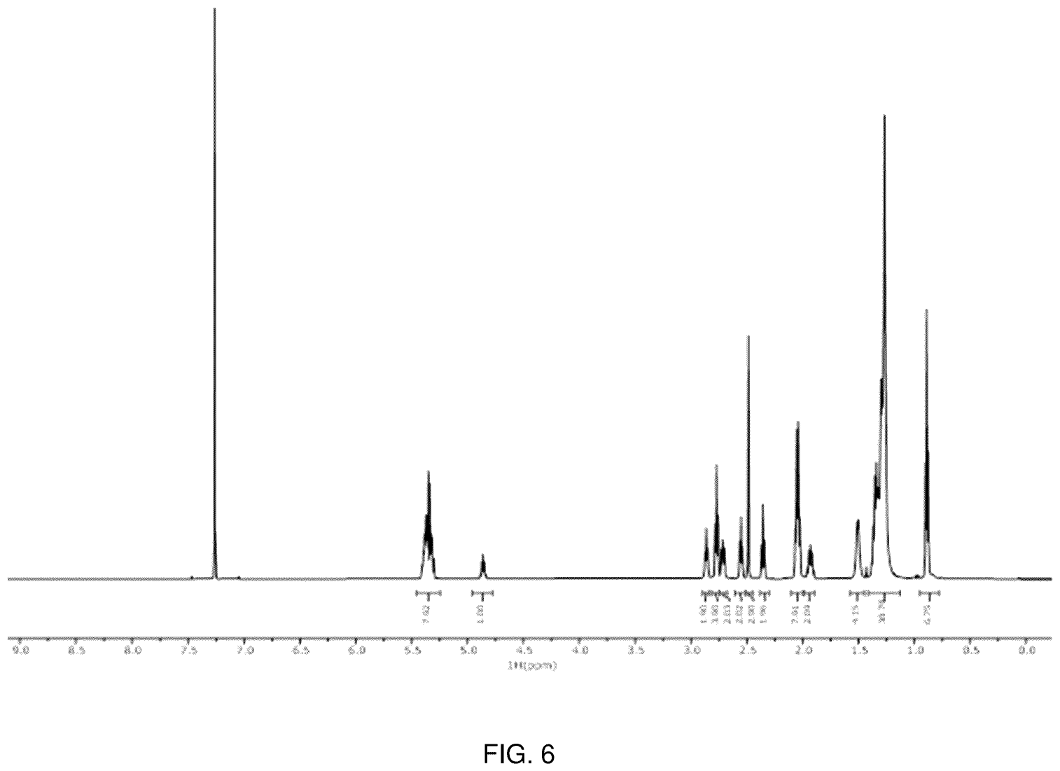

Compound ZW-A-CB2: To a 2 mL autosampler vial was added compound S3 (102 mg), acrylic acid (38.4 mg), N,N-diisopropylethylamine (2.4 mg) and 0.3 mL of ethanol. The mixture was stirred at 37° C. for 30 h, then diluted with DCM, washed with water and brine. The extract was dried over Na₂SO₄, concentrated, and purified via silica gel flash column chromatography (1%-20% MeOH in DCM/ethyl acetate 1:1) to yield compound ZW-A-CB2 as a light yellow oil. Yield: 86 mg. ¹H NMR (CDCl3) of compound ZW-A-CB2 is shown in FIG. 6.

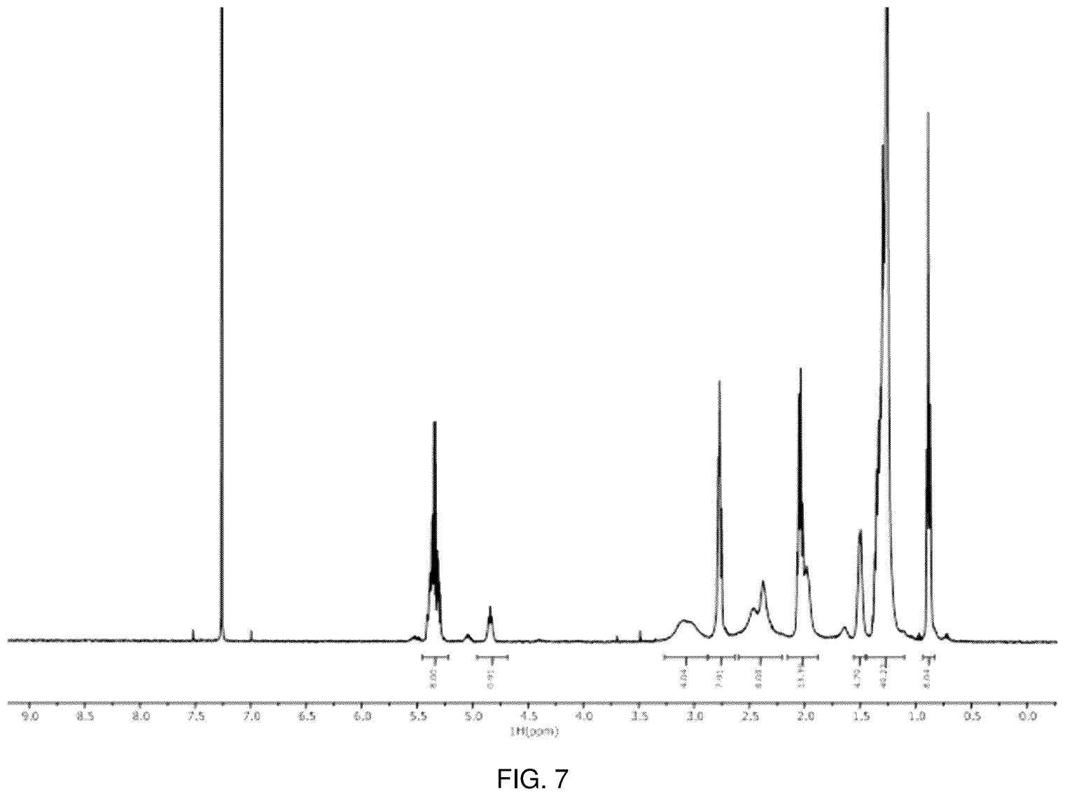

Compound ZW-A-CB3: To a 2 mL autosampler vial was added compound S3 (99.4 mg), t-butyl 4-bromobutyrate (46.8 mg), K₂CO₃ (89.6 mg), KI (8 mg), and a mixture of anhydrous acetonitrile (0.3 mL) and THE (0.1 mL). The mixture was stirred at 40° C. for 18 h, then diluted by DCM and filtered. The filtrate was concentrated and purified via silica gel flash column chromatography (1%-15% MeCN in DCM) to yield compound 3′ as a colorless oil. Yield: 95 mg. Compound 3′ (90 mg) was dissolved in a 50 v/v % solution of TFA in DCM and stirred at room temperature for 3 h. The reaction mixture was concentrated, then purified via silica gel flash chromatography using 1-15% MeOH in DCM to yield compound ZW-A-CB3 as a gummy liquid. Yield: 54 mg. ¹H NMR (CDCl3) of compound ZW-A-CB3 is shown in FIG. 7.

Reagents from commercial sources were used without further purification unless otherwise stated. All reactions were performed under a N₂ or argon atmosphere unless specified otherwise. Column chromatography was performed on a Biotage Isolera system using SiliCycle SiliaSep HP flash cartridges. NMR spectra were recorded using a Bruker or Varian 400 or 500 MHz spectrometer. All ¹H NMR experiments are reported in 6 units, parts per million (ppm), and were measured relative to the signals for residual chloroform (7.26 ppm) in deuterated solvent.

### Example 5 Synthesis and Characterization of ZW-B-CBn (n=1-3)

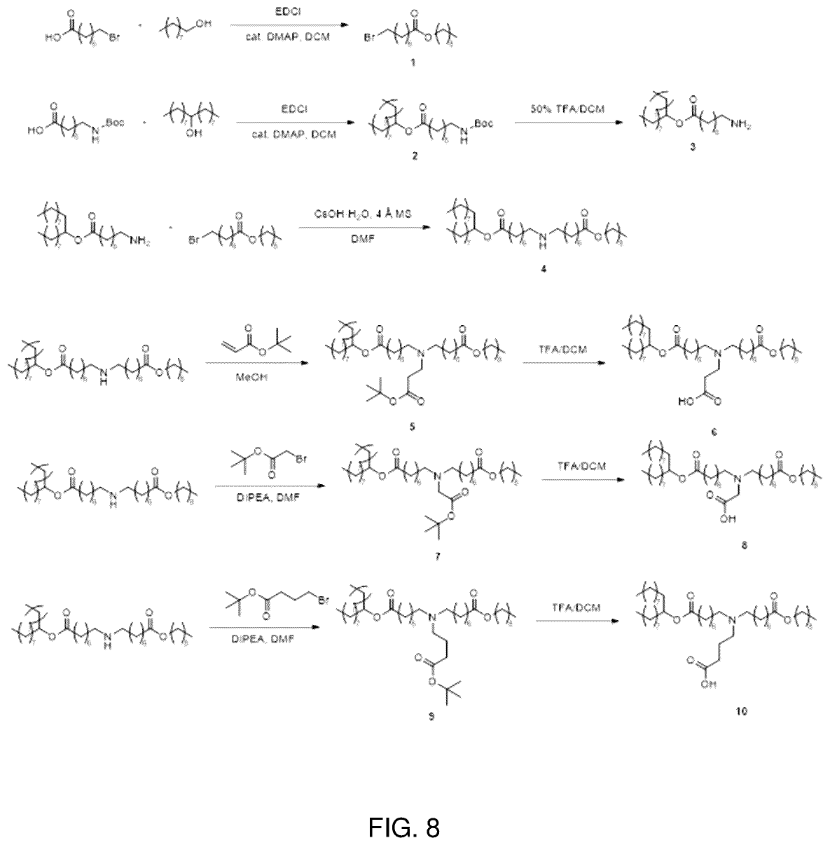

In this example, ZW-B-CBn (n=1,2,3) was synthesized and characterized in ¹H-NMR. The synthesis method of ZW-B-CBn is illustrated in FIG. 8 and described below.

Nonan-2-yl 8-bromooctanoate (1). A 100 mL round bottom flask was charged with 8-bromoacetic acid (2.00 g), nonan-1-ol (2.59 g), and 20 mL of dichloromethane. The flask was purged with N₂, and 1-(3-dimethylaminopropyl)-3-ethylcarbodiimide hydrochloride (2.25 g) and 4-dimethylaminopyridine (0.22 g) in 20 mL dichloromethane were added dropwise. The mixture was stirred under N₂ for 18 hours. Water was added, and the crude product was extracted with dichloromethane, washed with brine, and dried with sodium sulfate. The product was purified via column chromatography on silica gel using dichloromethane/hexanes as the eluent. Yield was 2.36 g (75%).

Heptadecan-9-yl 8-((tert-butoxycarbonyl)amino)octanoate (2). A 100 mL round bottom flask was charged with 8-((tert-butoxycarbonyl)amino)octanoic acid (1.04 g) and heptadecane-9-ol (1.13 g) with 20 mL of dichloromethane. The flask was purged with N₂, and 1-(3-dimethylaminopropyl)-3-ethylcarbodiimide hydrochloride (1.01 g) and 4-dimethylaminopyridine (0.05 g) in 20 mL dichloromethane were added dropwise. The mixture was stirred under N₂ for 20 hours. Water was added, and the crude product was extracted with dichloromethane, washed with brine, and dried with sodium sulfate. The product was purified via column chromatography on silica gel using ethyl acetate/hexanes as the eluent. Yield was 1.42 g (72%).

Heptadecan-9-yl 8-aminooctanoate (3). To a 20 mL vial was added heptadecan-9-yl 8-((tert-butoxycarbonyl)amino)octanoate (0.13 g), 2 mL of dichloromethane, and 2 mL of trifluoroacetic acid. The mixture was stirred for 3 hours. After removal of volatiles in vacuo, the crude product was dissolved in ethyl acetate, washed with ¹N NaOH and brine, and dried with sodium sulfate. Yield was 93.2 mg (93%).

Nonyl 8-((8-(octadecan-9-yloxy)-8-oxooctyl)amino)octanoate (4). A flame dried 50 mL Schlenk reaction was charged with activated 4 Å molecular sieves (500 mg), cesium hydroxide monohydrate (188 mg), and 6 mL of anhydrous dimethylformamide. The reaction was stirred for 10 minutes under N₂. Heptadecan-9-yl 8-aminooctanoate (500 mg) was added, and the mixture was stirred for an additional 30 minutes under N2. Nonan-2-yl 8-bromooctanoate (483 mg) was then added dropwise to the suspension, and the reaction was stirred for 24 hours under N₂. The mixture was filtered, poured into ¹N NaOH, extracted with dichloromethane, washed with brine, and dried with sodium sulfate. The product was purified via column chromatography on silica gel using methanol/chloroform as the eluent. Yield was 705 mg (84%).

Heptadecan-9-yl 8-((3-(tert-butoxy)-3-oxopropyl)(8-(nonan-2-yloxy)-8-oxooctyl)amino)octanoate (5). To a 1.5 mL vial was added nonyl 8-((8-(octadecan-9-yloxy)-8-oxooctyl)amino)octanoate (50 mg) and t-butyl acrylate (10.6 mg) in 0.5 mL methanol. The reaction was stirred under N₂ for 20 hours. Volatiles were removed in vacuo. Yield was 58 mg (98%).

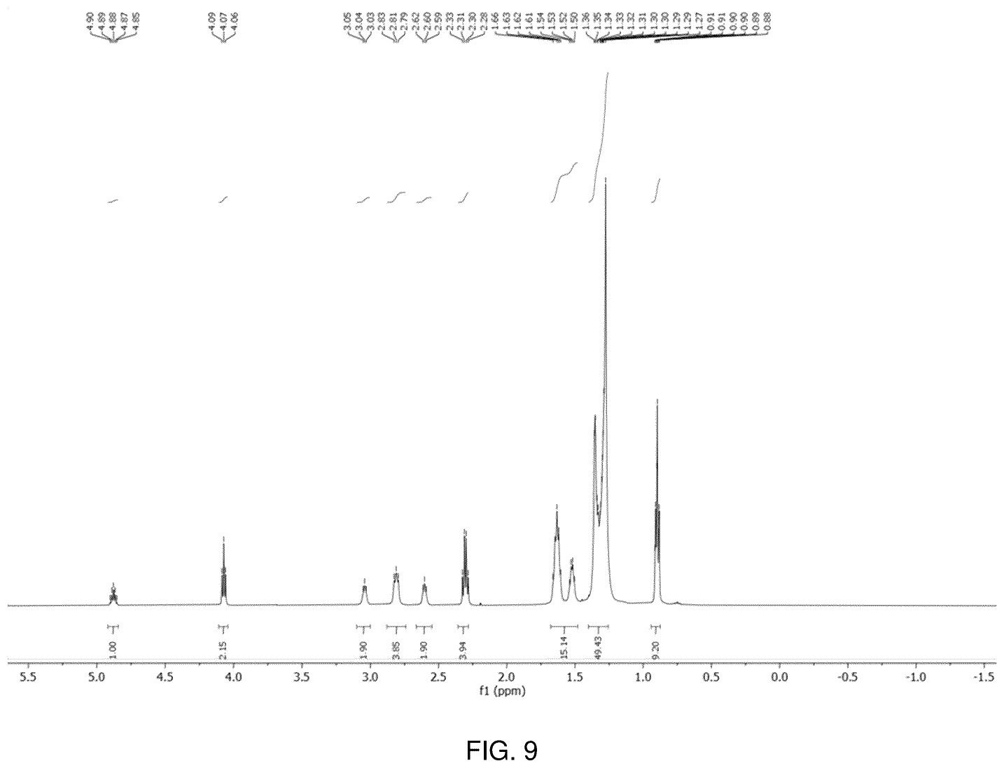

3-((8-(nonyloxy)-8-oxooctyl)(8-(octadecan-9-yloxy)-8-oxooctyl)amino)propanoic acid (6). To a 1.5 mL vial was added heptadecan-9-yl 8-((3-(tert-butoxy)-3-oxopropyl)(8-(nonan-2-yloxy)-8-oxooctyl)amino)octanoate (50 mg), 0.5 mL dichloromethane, and 0.5 mL trifluoroacetic acid. The mixture was stirred for 3 hours. Volatiles were removed in vacuo, and the product was purified via column chromatography on silica gel using methanol/dichloromethane as the eluent. Yield was 45 mg (94%). ¹H NMR spectrum of 3-((8-(nonyloxy)-8-oxooctyl)(8-(octadecan-9-yloxy)-8-oxooctyl)amino)propanoic acid (6) is shown in FIG. 9. This compound is named ZW-B-CB2.

Heptadecan-9-yl 8-((2-(tert-butoxy)-2-oxoethyl)(8-(nonan-2-yloxy)-8-oxooctyl)amino)octanoate (7). To a flame dried 1.5 mL vial was added nonyl 8-((8-(octadecan-9-yloxy)-8-oxooctyl)amino)octanoate (100 mg), t-butyl bromoacetate (117 mg), N,N-diisopropylethylamine (97 mg), and 1 mL anhydrous dimethylformamide. The reaction was stirred for 24 hours under N₂. The mixture was poured into ¹N NaOH, extracted with dichloromethane, washed with brine, and dried with sodium sulfate. The product was purified via column chromatography on silica gel using methanol/dichloromethane/ammonia as the eluent. Yield was 92 mg (79%).

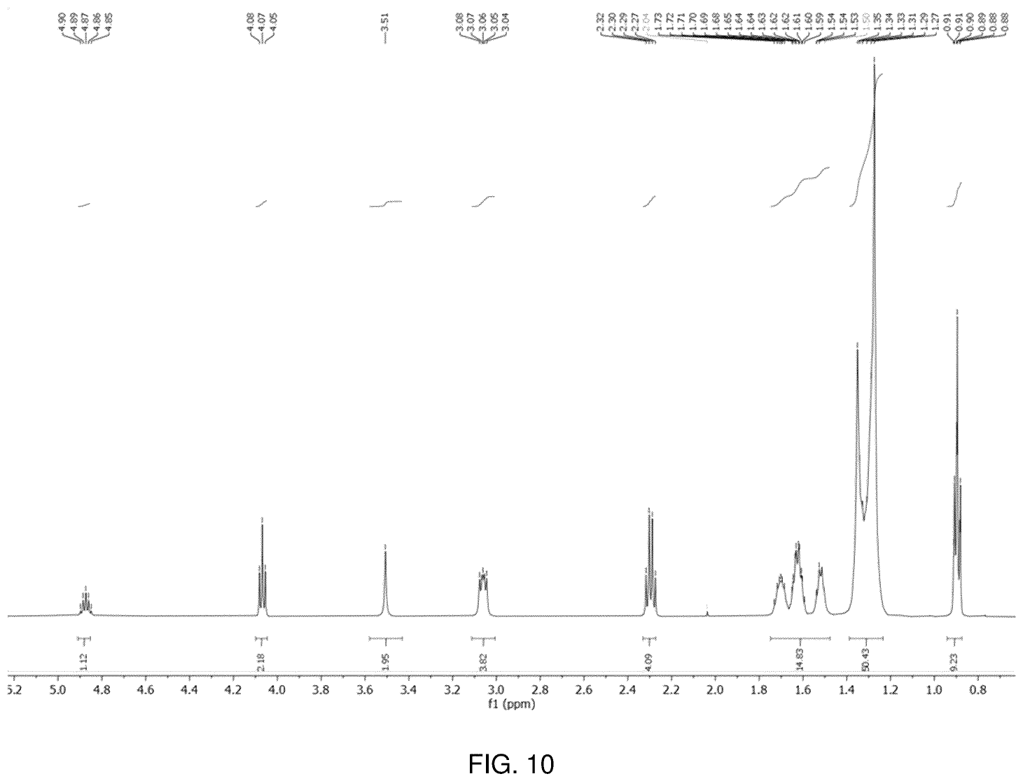

N-(8-(nonyloxy)-8-oxooctyl)-N-(8-(octadecan-9-yloxy)-8-oxooctyl)glycine (8). To a 1.5 mL vial was added heptadecan-9-yl 8-((2-(tert-butoxy)-2-oxoethyl)(8-(nonan-2-yloxy)-8-oxooctyl)amino)octanoate (50 mg), 0.5 mL dichloromethane, and 0.5 mL trifluoroacetic acid. The mixture was stirred for 3 hours. Volatiles were removed in vacuo, and the product was purified via column chromatography on silica gel using methanol/dichloromethane as the eluent. Yield was 42 mg (88%). 1H NMR spectrum of N-(8-(nonyloxy)-8-oxooctyl)-N-(8-(octadecan-9-yloxy)-8-oxooctyl)glycine (8) is shown in FIG. 10. This compound is named ZW-B-CB1.

Heptadecan-9-yl 8-((4-(tert-butoxy)-4-oxobutyl)(8-(nonan-2-yloxy)-8-oxooctyl)amino)octanoate (9). To a flame dried 1.5 mL vial was added nonyl 8-((8-(octadecan-9-yloxy)-8-oxooctyl)amino)octanoate (100 mg), t-butyl 4-bromobutyrate (134 mg), N,N-diisopropylethylamine (97 mg), and 1 mL anhydrous dimethylformamide. The reaction was stirred for 24 hours under N₂. The mixture was poured into 1 N NaOH, extracted with dichloromethane, washed with brine, and dried with sodium sulfate. The product was purified via column chromatography on silica gel using methanol/dichloromethane/ammonia as the eluent. Yield was 98 mg (81%).

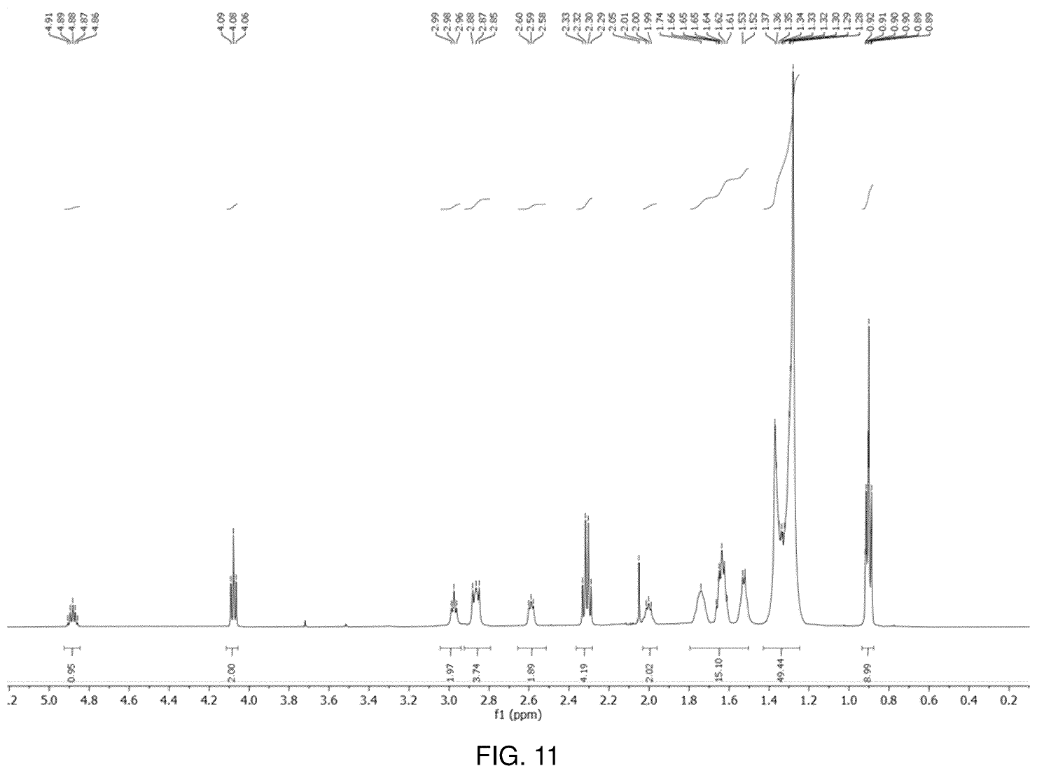

4-((8-(nonyloxy)-8-oxooctyl)(8-(octadecan-9-yloxy)-8-oxooctyl)amino)butanoic acid (10). To a 1.5 mL vial was added heptadecan-9-yl 8-((4-(tert-butoxy)-4-oxobutyl)(8-(nonan-2-yloxy)-8-oxooctyl)amino)octanoate (50 mg), 0.5 mL dichloromethane, and 0.5 mL trifluoroacetic acid. The mixture was stirred for 3 hours. Volatiles were removed in vacuo, and the product was purified via column chromatography on silica gel using methanol/dichloromethane as the eluent. Yield was 44 mg (93%). ¹H NMR spectrum of 4-((8-(nonyloxy)-8-oxooctyl)(8-(octadecan-9-yloxy)-8-oxooctyl)amino)butanoic acid (10) is shown in FIG. 11. This compound is named ZW-B-CB3.

### Example 6. Synthesis and Characterization of ZW-A-SulfoAmid-3

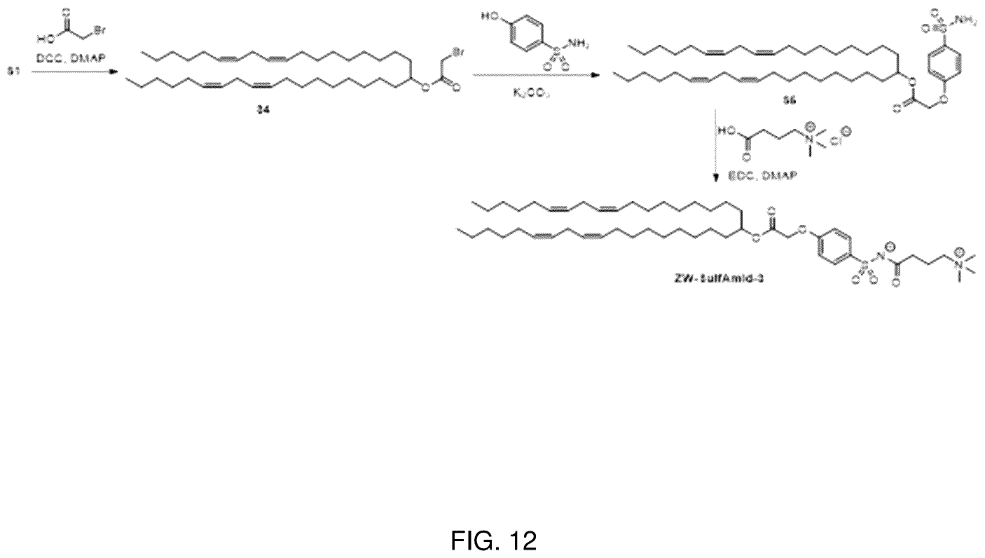

In this example, ZW-A-SulfAmid-3 was synthesized and characterized by ¹H-NMR. The synthesis method of ZW-A-SulfAmid-3 is illustrated in FIG. 12 and described below.

S4: To a 50 mL round bottom flask was added compound S1 (600 mg), bromo acetic acid (178 mg), DMAP (6 mg) and 5 mL of DCM. The mixture was cooled in an ice bath, and a 5 mL solution of DCC (178 mg) was added dropwise. The reaction was allowed to warm to room temperature and stirred over 12 h, then filtered, concentrated, and purified via silica gel flash chromatography (5%-30% DCM in hexanes). Compound S4 was obtained as a colorless oil. Yield: 0.59 g.

S5: To a 25 mL round bottom flask was added 4-Hydroxybenzenesulfonamide (60.8 mg), K₂CO₃ (43.4 mg), and 0.5 mL of DMF. The mixture was stirred for 5 min, then a 1 mL solution of S4 (121 mg) in DMF was added. The reaction mixture was stirred at 35° C. for over 20 h, then diluted with 5 mL DCM and filtered. The filtrate was concentrated and purified via silica gel flash chromatography (10%-40% ethyl acetate in hexenes) to yield S5 as a colorless oil. Yield: 102 mg.

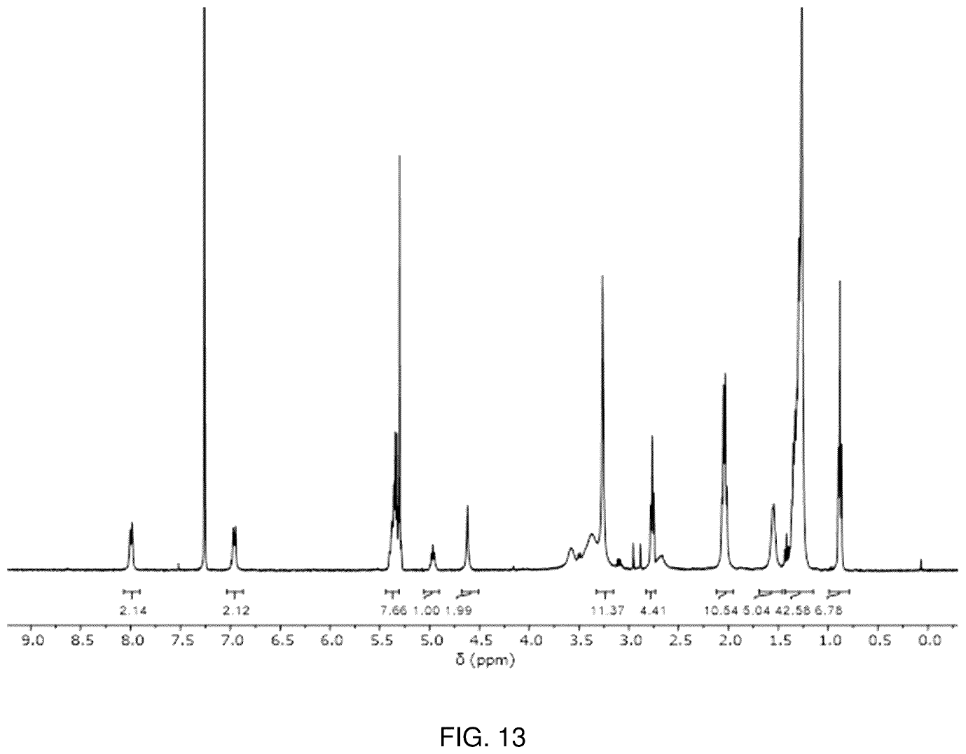

ZW-A-SulfAmid-3: To a 20 mL vial was added S5 (100 mg), 3-Carboxy-N,N,N-trimethylpropan-1-aminium chloride (48 mg), N′-ethylcarbodiimide hydrochloride (75 mg), DMAP (32 mg), triethyl amine (55 μL), and 2 mL of DMF. The reaction mixture was stirred at RT overnight, then purified via silica gel flash chromatography (5%-50% MeOH in DCM) to yield the title compound as a light yellow solid. Yield: 55 mg. 1H NMR (CDCl3) of compound ZW-A-SulfAmid-3 is shown in FIG. 13.

### Example 7. Preparation and In Vitro Characterization of PCB-LNP Containing ZW-B-CB2 (Non-PEG Formulations)

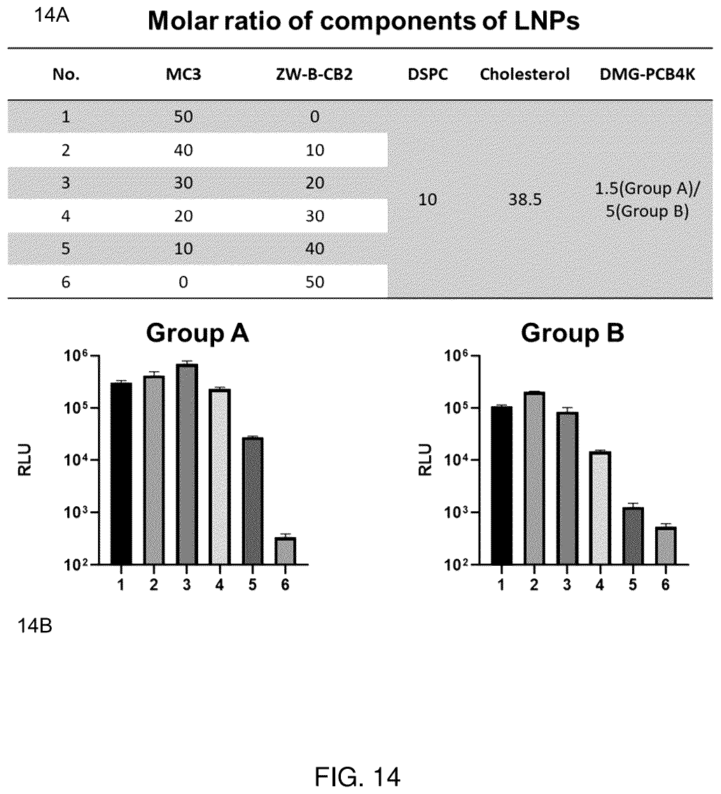

In this example, ZW-B-CB2 synthesized from Example 5 was incorporated PCB-LNPs described in Example 2 at different ratio to replace MC3. Other components included DSPC, cholesterol and DMG-PCB4K. The molar ratio of each component is listed in FIG. 14.

Lipids were dissolved in ethanol and mRNA was dissolved in a 50 mM citric buffer (pH3). Encapsulation of mRNA in LNPs was prepared by mixing two phases at a ratio of 1:3 (ethanol: aqueous, v/v %) while stirring vigorously. Formulations were dialyzed against PBS (pH 7.4) in a dialysis cassette for at least 10 hours, concentrated by passing through a 0.22-μm filter, and stored at 4° C. until use.

In vitro transfection of LNPs encapsulating mRNA encoding Fluc was performed to evaluate the transfection efficiency of the formulations. Briefly LNPs were transfected to HepG2 (ATCC No. HB-8065) and luciferase expression was analyzed at 6-h after transfection. Cell culture conditions were described in Example 2. During transfection, RNA was added 100 ng per well in triplicates. After 6 h of transfection, the culture medium containing LNPs was carefully removed, and the cells were rinsed once with PBS gently. Luciferase expression was measured using a luciferase assay (Promega Cat #: E1501) following the manufacture's protocol.

### Example 8. In Vivo Multiple Systemic Deliveries of PCB-LNPs Containing ZW-B-CBn (n=1-3) (Non-PEG Formulations)

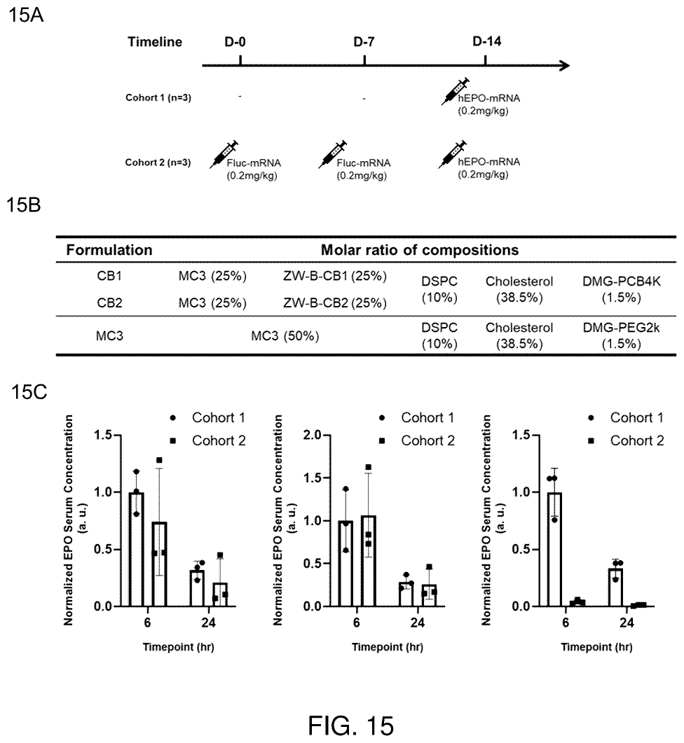

Accelerated blood clearance (ABC) effect has been reported in cases where LNPs were injected multiple times with rapid clearance of LNPs observed in the subsequent injections. ABC effect is largely caused by antibodies generated against lipid components of the LNPs which have been reported to be immunogenic. In this example, PCB-LNP containing ZW-B-CBn from Example 7 were used to study the ABC effect of LNPs. MC3-based PEG-LNP from Example 2 was used as a control. Compositions of each formulation is listed in FIG. 15b.

For in vivo pharmacokinetic (PK) study, male C57BL/6J mice aged 3-4 weeks in groups of three were administered intravenously with mRNA-LNP at a dosage of 5 μg of mRNA. Two different injection cohorts were performed. (FIG. 15a) In cohort 1, only one dose of hEPO-mRNA (human erythropoietin mRNA) was given. In cohort 2, Fluc-mRNA (fly luciferase mRNA) was dosed for the first two injections, and hEPO-mRNA for the last injection. (FIG. 15a) Cohort 2 was designed in a way to eliminate anti-hEPO immune response induced from first two injections, and thereby the PK profile of hEPO in the third injection is mainly indicating the ABC effect due to LNP components. To determine the PK profile of hEPO, mice sera were collected at 6-hour, 24-hour post-injection, and analyzed using ELISA (DuoSet, hEPO ELISA kit, R&D).

As shown in FIG. 15c, LNPs containing PCB and ZW-B-CB1/ZW-B-CB2 showed less ABC effect in injection cohort 2 compared to MC3 formulations. The results indicate that PCB-LNPs with ZW-B-CBn induced reduced ABC effects and therefore were less immunogenic.

### Example 9. In Vivo Systemic Delivery of PCB-LNP Containing ZW-B-CB2 and

### Targeting Phosphoserine (PS) Lipids (Non-PEG Formulations)

In this example, a negatively charged phospholipid, 1,2-dioleoyl-sn-glycero-3-phospho-L-serine (DOPS), was incorporated into the PCB-LNPs formulation described in Example 7. Briefly, the molar ratio of MC3:ZW-B-CB2:DSPC:Cholesterol:DMG-PCB4k:DOPS equals to 20:30:10:38.5:1.5:5. The formulation with DOPS is named PS-PCB-mLNP (i.e., PCB-LNP containing ZW-B-CB2 and PS lipids), whereas the one without DOPS is named PCB-mLNP (i.e., PCB-LNP containing ZW-B-CB2 lipid) as a control. Lipid mixtures and mRNA aqueous solutions were rapidly mixed in a microfluidic channel as described in Example 2, and then washed with PBS to generate final products. DOPS was used as a non-cationic lipid to target to the secondary lymphoid organs (SLOs).

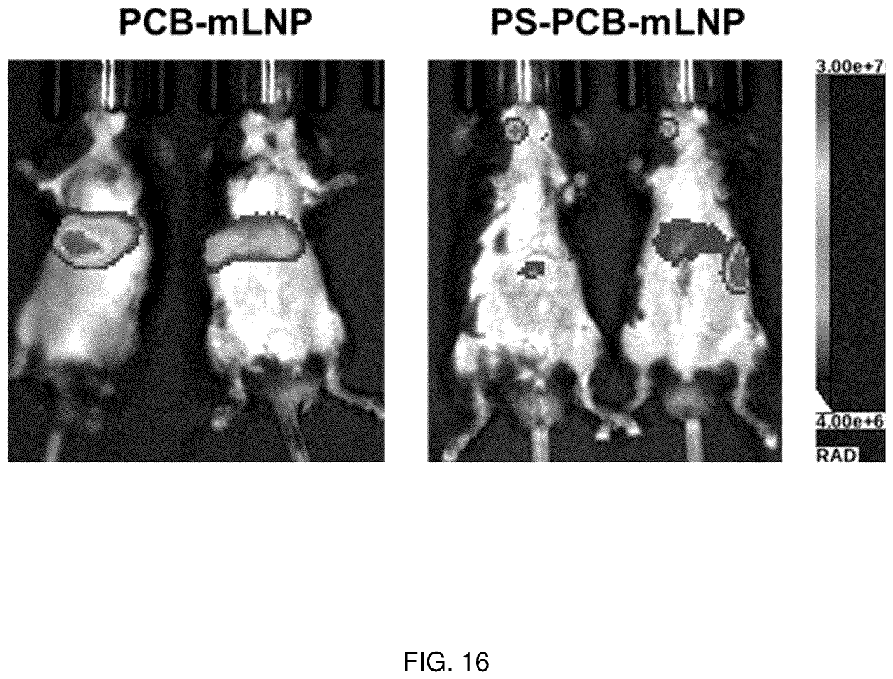

To study the protein expression in vivo, the PS-PCB-mLNP carrying Fluc-encoding mRNA was delivered to the mouse via intravenous injections. Bioluminescent images were taken 6 hours post-administration using IVIS as described above. As shown in FIG. 16, the IVIS image shows mRNA expression shifted from the liver towards the spleen and superficial lymph nodes. In the control group, the Fluc expression mostly occurred in the liver for PCB-mLNPs without the targeting lipid DOPS. Therefore, the incorporation of DOPS allowed more protein expression in the spleen and lymph nodes, indicating a targeting effect to these SLOs. The result demonstrates PS-PCB-mLNP as a promising platform for in vivo mRNA delivery to SLOs for applications including immunotherapy and vaccines.

### Example 10. Preparation and In Vitro Characterization of PEG LNPs Containing

ZW-B-CBn (n=1-3) (PEG Formulations)

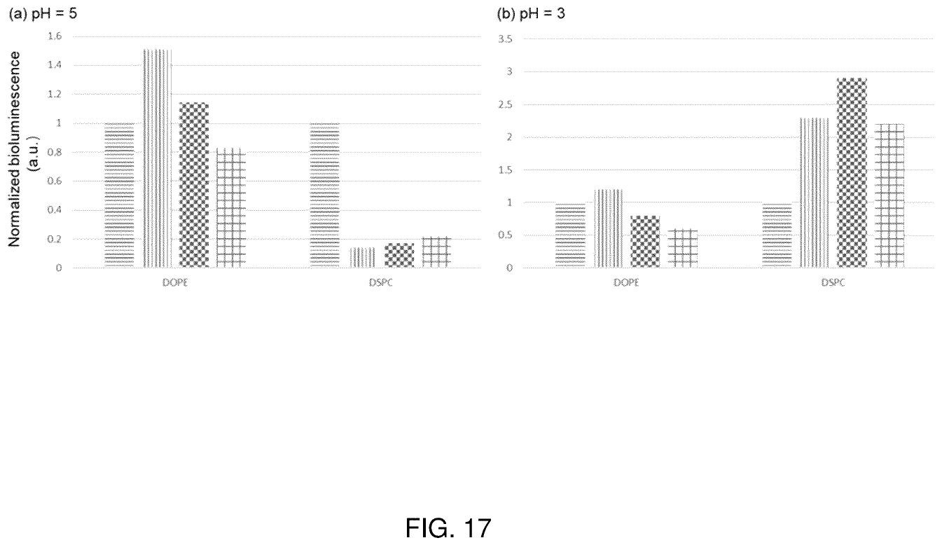

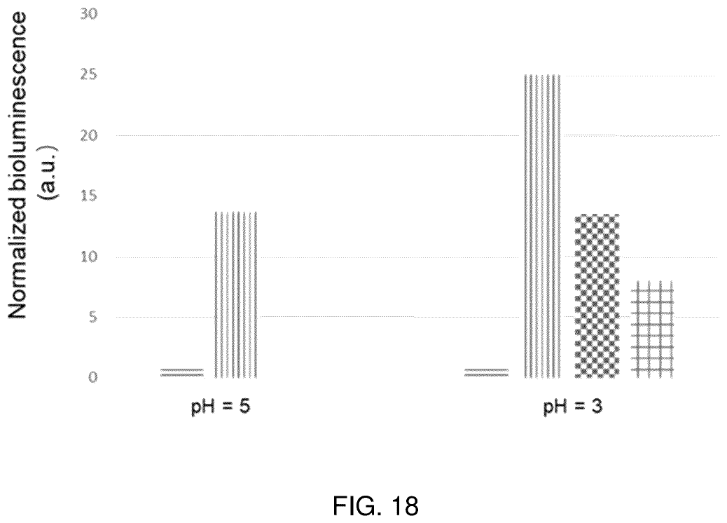

It has been reported that ionizable lipids, the major component in LNPs (or denoted as PEG LNPs to distinguish PCB LNPs where PEG lipid is replaced by PCB lipid), are immunogenic. To reduce the immunogenicity, ionizable lipids are partially substituted by ZW-B-CBn obtained from example 6 to generate formulations. Briefly, three conditions varying different helper lipids were tested: 1) DSPC, 2) DOPE and 3) No helper lipid. For conditions 1 and 2, LNPs composed of the following lipids: MC3+ZW-B-CBn, DSPC or DOPE, cholesterol and DMG-PEG2k at a molar ratio of 50:10:38.5:1.5. For condition 3, LNPs composed of the following lipids: MC3+ZW-B-CBn, cholesterol and DMG-PEG2k at a molar ratio of 50:38.5:1.5. In vitro transfection results are shown in FIG. 17 and FIG. 18. Preparation and in vitro transfection of the LNPs are described below.

Lipids were dissolved in ethanol at indicated molar ratio and mRNA was dissolved in a 20 mM sodium acetate buffer (pH 5) or a 20 mM citric buffer (pH 3). To generate LNPs, ethanol and aqueous solutions were rapidly mixed at a ratio of 1:3 (ethanol:aqueous) while stirring vigorously. Formulations were dialyzed against PBS (pH 7.4) in a dialysis cassette for at least 10 hours, concentrated by passing through a 0.22-μm filter, and stored at 4° C. until use. All formulations were tested for particle size, and RNA encapsulation.

In vitro transfection of LNPs encapsulating mRNA encoding Fluc was performed to evaluate the transfection efficiency of the formulations. Briefly, LNPs were transfected to HepG2 (ATCC No. HB-8065) and luciferase expression was analyzed at 6-h after transfection. Cell culture conditions were described in example 2. During transfection, RNA was added 100 ng per well in triplicates. After 6 h of transfection, the culture medium containing LNPs was carefully removed, and the cells were rinsed once with PBS gently. Luciferase expression was measured using a luciferase assay (Promega Cat #: E1501) following the manufacture's protocol.

### Example 11. Preparation and In Vitro Characterization of PEG LNPs Containing PS Lipid as a Targeting Lipid (PEG Formulations)

In this example, a negatively charged phospholipid, 1,2-dioleoyl-sn-glycero-3-phospho-L-serine (DOPS), was incorporated into the MC3-based formulation to generate PS5-LNP. Briefly, PS5-LNPs were generated using a microfluidic channel described in example 2, which contains MC3, DSPC, Cholesterol, DMG-PEG2k, and DOPS at a molar ratio of 50:10:38.5:1.5:5. LNPs without DOPS were used as control (PSO-LNP).

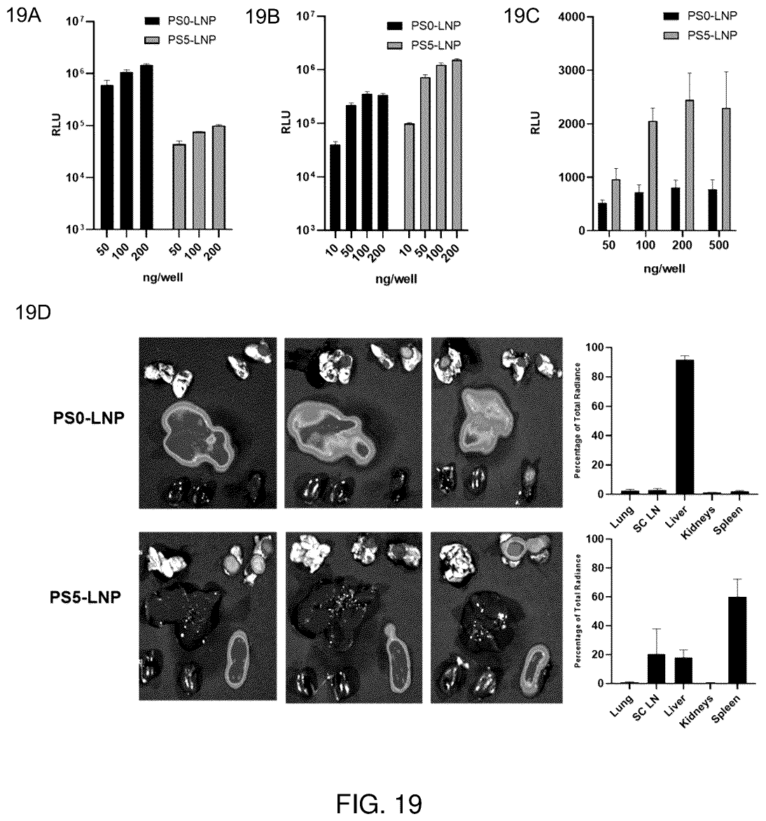

Both PS5-LNP and PSO-LNP encapsulated mRNA encoding firefly's luciferase (Fluc) and transfected to HepG2 (ATCC No. HB-8065) and Raw264.7 (ATCC No. TIB-71) to evaluate transfection efficiency in vitro. For cell culture conditions, HepG2 was maintained at 37° C. with 5% CO₂ in Eagle's Minimum Essential Medium (EMEM) supplemented with 10% fetal bovine serum (FBS) and 1× Penicillin-Streptomycin (Pen-Strep). Similarly, Raw264.7 was maintained in the same culturing conditions in Dulbecco's Modified Eagle's Medium (DMEM) supplemented with 10% FBS and 1× Pen-Strep. To transfect LNPs into the cell, different amounts of RNA (as indicated in the plot) were added to the cells plated in a 96-well plate seeded at 60-70% confluency. After 6 h of transfection, the culture medium containing PS-LNPs was carefully removed, and the cells were rinsed once with PBS gently. 30 μL of Passive Lysis Buffer (Promega Cat #: E1941) was added to lyse the cells and 100 μL of D-luciferin substrate (Promega Cat #: E1501) was added to each well. Immediately after adding the substrate, the luminescence intensity was measured in a white opaque plate using a Cytation 5 microplate reader (Biotek, USA). As shown in FIG. 19, PS5-LNPs possess transfection efficiency in both HepG2 (FIG. 19a), a hepatocyte cell line, and RAW264.7 (FIG. 19b), a monocyte/macrophage cell line. In addition, compared to PSO-LNP, PS5-LNP show higher mRNA expression level in monocytes/macrophages but much lower in hepatocytes. In addition, PS5-LNP also had higher transfection efficiency in primary mouse splenocytes (FIG. 19c). The difference of protein expression in these cells indicates that PS5-LNP has the potential to selectively deliver mRNA to immune cells but not liver cells.

The discovery from in vitro studies was corroborated in vivo. Both PS5-LNP and PSO-LNP were encapsulated mRNA encoding firefly luciferase (Fluc) and delivered to the C57BL/6 mice (0.2 mg/kg) via retro-orbital injections. At 6 hour post-administration, mice were euthanized and different organs were isolated to analyze the mRNA expression level. For PS5-LNP, 40% of total bioluminescent signals was found in cervical lymph nodes and spleens. On the contrary, for control group PSO-LNP, more than 90% of bioluminescent signal was found in the liver but few in lymph nodes and spleens (FIG. 19d). The result clearly concludes a biodistribution shift towards SLOs after incorporation of DOPS. It is noteworthy that addition of PS target to lymph node and spleen without sacrificing the overall transfection efficiency, while many other reported that negatively charged lipid reduce the transfection efficiency of the particle.

### Example 12—Prophetic Example. Preparation and Characterization of PCB-LNP with Reduced Components

A library of PCB-LNPs with a reduced amount of cholesterol will be generated. As shown in Table 1, the ratio of cholesterol will be ranged from 0 to 38.5 mol %, and correspondingly, DMG-PCB will be ranged from 1.5 to 40 mol %. The amount of ionizable lipids (50 mol %) and helper lipids (10 mol %) remain unchanged as described in Example 2.

TABLE 1
Experimental design for PCB-LNP with reduced cholesterol

| Number | Cholesterol (mol %) | DMG-PCB (mol %) |
|---|---|---|
| 1 | 0 | 40 |
| 2 | 10 | 30 |
| 3 | 20 | 30 |
| 4 | 30 | 10 |
| 5 | 38.5 | 1.5 |

Each formulation will be prepare following the same procedure described in Example 2. Characterization of size, zeta-potential and encapsulation efficiency will be conducted for each formulation. For efficacy test, each formulation will be transfected in vitro to evaluate the transfection efficiency. Specifically, an mRNA encoding firefly luciferase (Fluc) will be encapsulated by each formulation, respectively. HEK 293T (ATCC, CRL-11268) will be seeded at 4×104 cells/well into 96-well plates in 100 μL of culture medium (DMEM with 10% FBS) and allowed to attach overnight in 37° C. with 5% CO₂. Then, 5 μL of Fluc mRNA-loaded LNPs were added to the medium and incubated for 6 hours. Each formulation will have five replicates, and PBS will be added as a negative control. After transfection, culture medium will be removed carefully, and the expression of Fluc will be evaluated using a Luciferase Assay System.

Moreover, the best performing formulation will be delivered into mice to evaluate the in vivo expression and biodistribution. Specifically, Fluc mRNA-loaded LNP (1 mg/kg of mRNA) will be injected into the male C57BL/6J mice aged 6-7 weeks. After 6 hours of transfection, mice will be administered an intraperitoneal injection of D-luciferin (30 mg/mL in PBS). After 10 minutes, the mice will be sacrificed, and eight organs will be collected (liver, spleen, kidneys, lungs, and spleens). The organs' luminescence will be analyzed using an optical imaging system and quantified using suitable software to measure the radiance of each organ in photons/sec.

### Example 13—Prophetic Example. Preparation and Characterization of LNP with or without PC Moiety

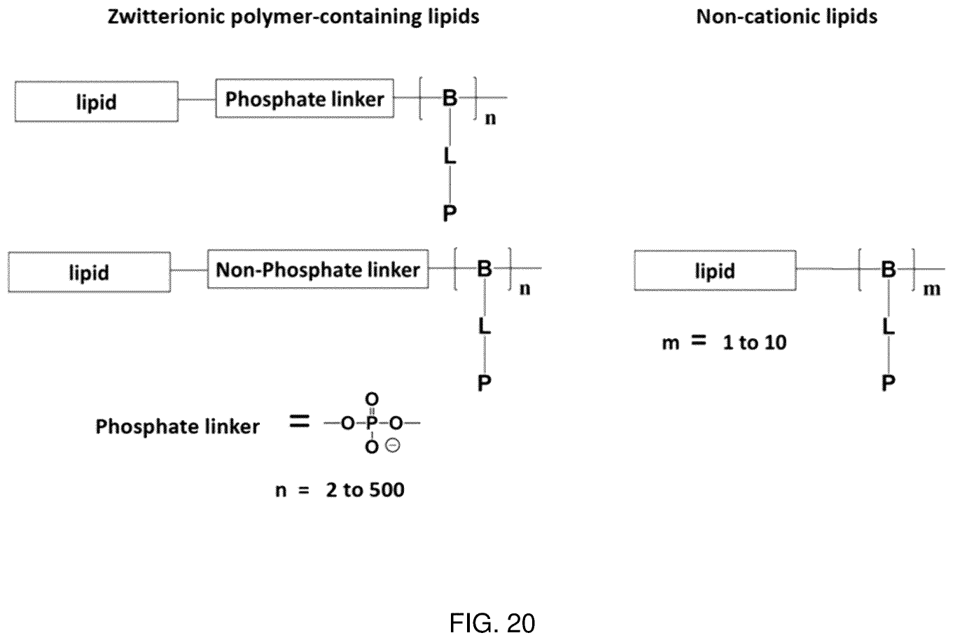

In this example, a formulation using molecules without PC moiety will be generated to address the immunogenicity from PC moiety. (FIG. 20.) In one formulation without PC moiety, four components of the LNPs include ionizable lipids, DMG-CB1, cholesterol and DMG-PCB. DMG-CB1 will be generated from Example 1 where CB will be attached to DMG-N to replace the helper lipid DSPC from previous formulations. In a formulation with PC moiety, four components of the LNPs include ionizable lipids, DSPC, cholesterol and DSPE-PCB. DSPE-PCB will be generated following the reference (Z. Cao, L. Zhang and S. Jiang, Superhydrophilic Zwitterionic Polymers Stabilize Liposomes, Langmuir, 28, 11625, 2012).

Each formulation will be prepared following the same procedure described in Example 2. Characterization of size, zeta-potential and encapsulation efficiency will be conducted for each formulation. For efficacy test, each formulation will be transfected in vitro to evaluate the transfection efficiency. Specifically, an mRNA encoding firefly luciferase (Fluc) will be encapsulated by each formulation, respectively. HEK 293T (ATCC, CRL-11268) will be seeded at 2×104 cells/well into 96-well plates in 100 μL of culture medium (DMEM with 10% FBS), and allowed to attach overnight in 37° C. with 5% CO₂. Then, 5 μL of Fluc mRNA-loaded LNPs were added to the medium and incubated for 6 h. Each formulation will have five replicates, and PBS will be added as a negative control. After transfection, the culture medium will be removed carefully, and the expression of Fluc will be evaluated using a Luciferase Assay System.

Furthermore, the best performing formulation will be delivered into mice to evaluate the in vivo expression and biodistribution. Specifically, Fluc mRNA-loaded LNP (1 mg/kg of mRNA) will be injected into the male C57BL/6J mice aged 6-7 weeks. After 6 hours of transfection, mice will be administered an intraperitoneal injection of D-luciferin (30 mg/mL in PBS). After 10 minutes, the mice will be sacrificed, and eight organs will be collected (liver, spleen, kidneys, lungs, and hearts). The organs' luminescence will be analyzed using an optical imaging system and quantified using suitable software to measure the radiance of each organ in photons/sec.

### Example 14—Prophetic Example. Combination of Lipid Components

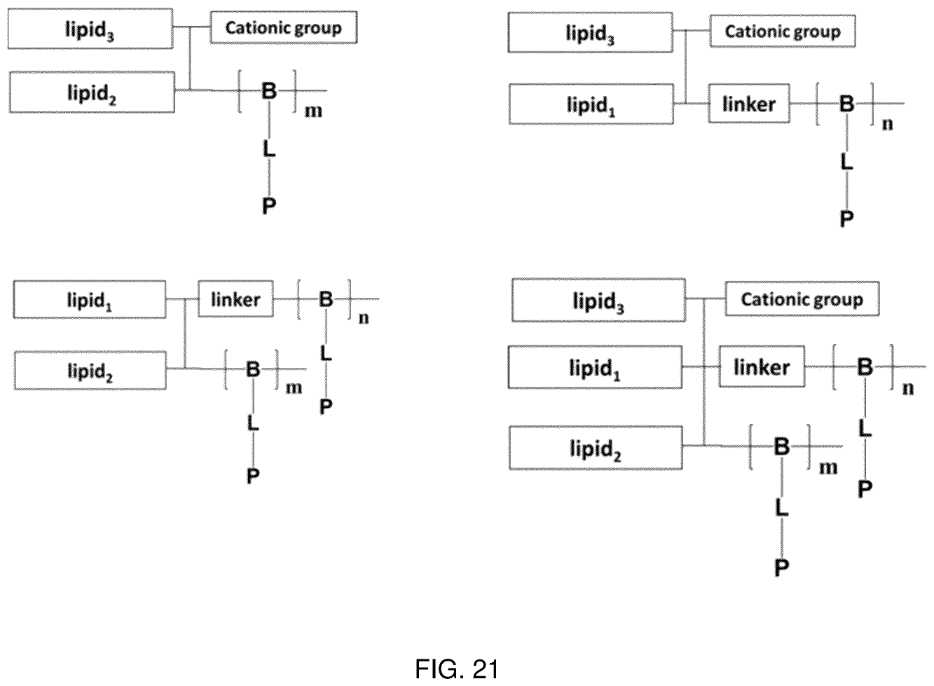

As also shown in FIG. 21, any lipid components (zwitterionic polymer modified lipids, cationic lipids, non-cationic lipids, and cholesterol or its derivative) can be chemically combined with any other components to achieve the compositions and methods described above.

While there have been shown and described what are at present considered the preferred embodiments of the invention, those skilled in the art may make various changes and modifications which remain within the scope of the invention defined by the appended claims.

What is claimed is:

1. A lipid nanoparticle composition comprising:
(i) at least one zwitterionic polymer-containing lipid in which a lipid moiety is covalently attached to a zwitterionic polymer;
(ii) at least one non-cationic lipid selected from charged and uncharged lipids, wherein the non-cationic lipid is not attached to a polymer;
(iii) at least one cationic or ionizable lipid containing a secondary, tertiary, or quaternary amino group and
(iv) at least one therapeutic substance.

2. The composition of claim 1, wherein said lipid moiety in component (i) is a diacylglyceride.

3. The composition of claim 1, wherein component (i) excludes a polyalkylene oxide segment.

4. The composition of claim 1, wherein the zwitterionic polymer in component (i) is selected from the group consisting of a poly(carboxybetaine) (PCB), a poly(sulfobetaine), a poly(phosphobetaine), poly(phosphatidylcholine), glutamic acid-lysine (EK)-containing polypeptide, a poly(trimethylamine N-oxide) polymer and a poly(zwitterionic phosphatidyl serine).

5. The composition of claim 1, wherein the zwitterionic polymer in component (i) is a betaine polymer.

6. The composition of claim 5, wherein the betaine polymer is a poly(carboxybetaine), poly(sulfobetaine), or poly(phosphobetaine) polymer.

7. The composition of claim 1, wherein the non-cationic lipid in component (ii) contains a zwitterionic moiety.

8. The composition of claim 7, wherein the zwitterionic moiety is selected from the group consisting of a phosphobetaine, phosphatidylcholine, carboxybetaine, sulfobetaine, trimethylamine N-oxide, glutamic acid-lysine (EK)-containing peptide, or zwitterionic phosphatidyl serine moiety.

9. The composition of claim 1, wherein the non-cationic lipid is selected from the group consisting of a dioleoylphosphatidylglycerol (DOPG), dipalmitoylphosphatidylglycerol (DPPG), dioleoylphosphatidylethanolamine (DOPE), palmitoyloleoyl-phosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl-phospho ethanolamine, 16-O-dimethyl-phosphoethanolamine, 18-1-trans-phosphoethanolamine, 1-stearoyl-2-oleoyl phosphatidyethanolamine (SOPE), and 1,2-dioleoyl-sn glycero-3-phophoethanolamine (transDOPE).

10. The composition of claim 1, wherein component (ii) excludes a polyalkylene oxide segment.

11. The composition of claim 1, wherein the non-cationic lipid in component (ii) is a phospholipid.

12. The composition of claim 11, wherein the phospholipid is a phosphatidyl serine lipid.

13. The composition of claim 1, wherein the cationic or ionizable lipid in component (iii) possesses a secondary, tertiary, or quaternary amino group.

14. The composition of claim 1, wherein the cationic or ionizable lipid in component (iii) possesses a secondary, tertiary, or quaternary group along with a functional group which is negatively charged under physiological conditions.

15. The composition of claim 14, where the cationic or ionizable lipid comprises: 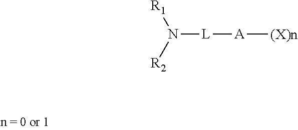
wherein:
R₁R₂═H or an alkyl group and wherein the alkyl group can be saturated, unsaturated, branched, and/or unbranched, and can further comprise one or more heteroatoms including but not limited to N, O, F, Si, P, S, Cl, Br, and F;
L comprises a covalent linker group between N and A, wherein the covalent linker group can comprise one or more of —CH₂—, —CH₂CH(OH)—, —CH₂CHClCH₂—, —CH₂OCH₂—, —CH₂SCH₂—, —CH₂SSCH₂—, —CH₂COOCH₂—, C, H, and can further comprise one or more heteroatoms including but not limited to N, O, F, Si, P, S, Cl, Br, and F; and
A-(X)n is a functional group that is negatively charged under certain pH conditions and can comprise:
(i) A(X)n is a carboxylic acid group (A=-COOH and n=O) or
(ii) A(X)n is a phosphate, where A= 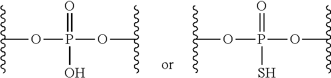 [SMILES: *OP(=O)(O)O* ; *OP(=O)(S)O*]
and n=1. X═H or an alkyl group that is saturated or unsaturated, branched or not branched, all-carbon or containing heteroatoms such as but not limited to N, O, F, Si, P, S, Cl, Br, and F or
(iii) A(X)n is a sulfonic acid group, where A= 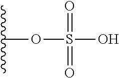 [SMILES: *OS(=O)(=O)O]
and n=0
(iv) A(X)n is a sulfonamide group, where A= 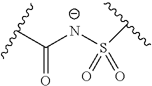 [SMILES: *C(=O)[N-]S(*)(=O)=O]
and n=1, X═H or an alkyl group that is saturated or unsaturated, branched or not branched, all carbon and hydrogen or containing heteroatoms such as but not limited to N, O, F, Si, P, S, Cl, Br, and F.

16. The composition of claim 14, where the functional group is selected from the group consisting of: 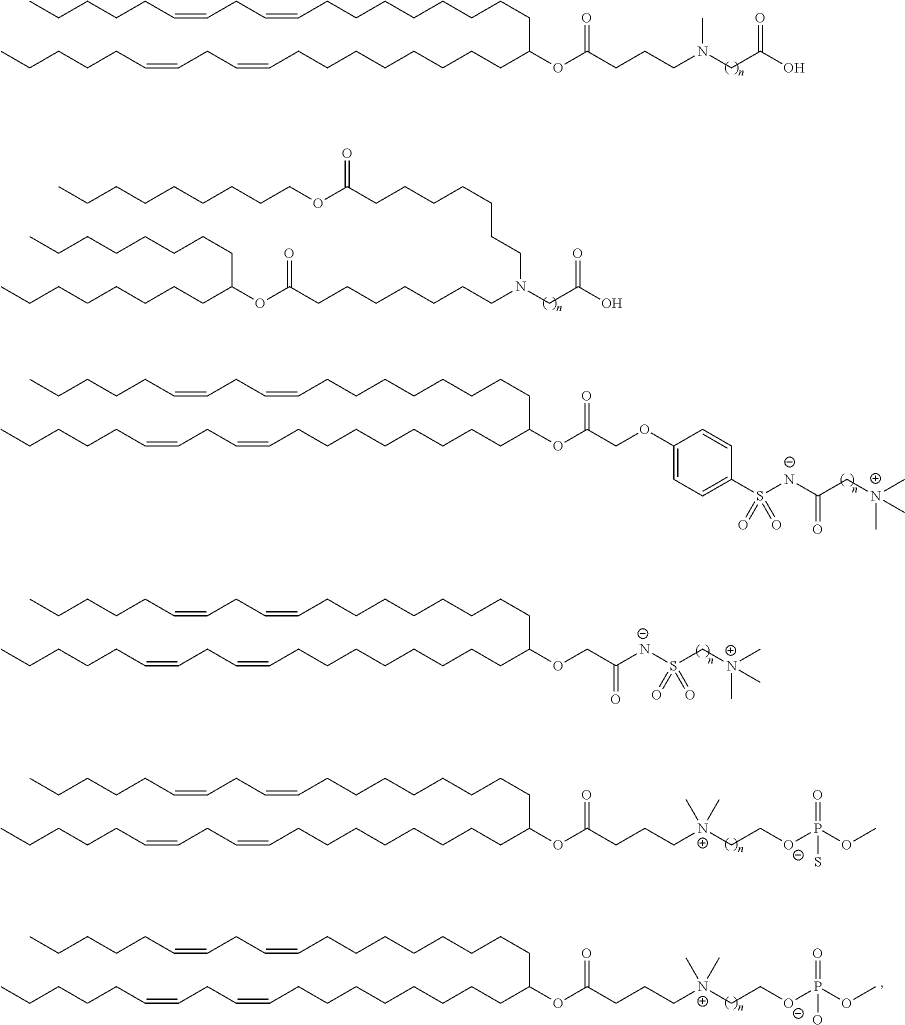
and
wherein n=1 to 10.

17. The composition of claim 1, wherein the cationic or ionizable lipid in component (iii) excludes a polyalkylene oxide segment.

18. The composition of claim 1, wherein the lipid nanoparticle composition further comprises: (v) cholesterol or derivative thereof.

19. The composition of claim 1, wherein the therapeutic substance in component (iv) is a nucleic acid molecule.

20. The composition of claim 19, wherein the nucleic acid molecule is an RNA.

21. The composition of claim 20, wherein the RNA is mRNA.

22. The composition of claim 20, wherein the RNA is viral mRNA.

23. The composition of claim 20, wherein the RNA is selected from the group consisting of message RNA (mRNA), small interfering RNA (siRNA), microRNA (miRNA), circularRNA (circRNA), long-noncoding RNA (lncRNA), antisense oligonucleotide (ASO), a CRISPR-related RNA, a Cas nuclease mRNA, a guide RNA, a single-guide RNA, and combinations thereof.

24. The composition of claim 20, wherein the therapeutic substance is a spike protein of a virus.

25. A method of delivering a therapeutic substance to a subject, the method comprising administering to said subject a lipid nanoparticle composition comprising:
(i) at least one zwitterionic polymer-containing lipid in which a lipid moiety is covalently attached to a zwitterionic polymer;
(ii) at least one non-cationic lipid selected from charged and uncharged lipids, wherein the non-cationic lipid is not attached to a polymer;
(iii) at least one cationic or ionizable lipid containing a secondary, tertiary, or quaternary amino group and
(iv) at least one therapeutic substance.

26. The method of claim 25, wherein the lipid nanoparticle composition is delivered to cells of the subject.

27. The method of claim 25, wherein the therapeutic substance is a nucleic acid molecule, and administration thereof results in gene therapy of the subject.

28. The method of claim 25, wherein the therapeutic substance is a nucleic acid molecule, and administration thereof results in vaccination of the subject.

29. The method of claim 25, wherein said lipid moiety in component (i) is a diacylglyceride.

30. The method of claim 25, wherein component (i) excludes a polyalkylene oxide segment.

31. The method of claim 25, wherein the zwitterionic polymer in component (i) is selected from the group consisting of a poly(carboxybetaine) (PCB), a poly(sulfobetaine), a poly(zwitterionic phosphobetaine), glutamic acid-lysine (EK)-containing polypeptide, poly(phosphatidylcholine), and a poly(trimethylamine N-oxide) polymer.

32. The method of claim 25, wherein the zwitterionic polymer in component (i) is a betaine polymer.

33. The method of claim 32, wherein the betaine polymer is a carboxy betaine polymer.

34. The method of claim 25, wherein the non-cationic lipid in component (ii) contains a zwitterionic moiety.

35. The method of claim 34, wherein the zwitterionic moiety is selected from the group consisting of a phosphobetaine, phosphatidylcholine, carboxybetaine, sulfobetaine, trimethylamine N-oxide, glutamic acid-lysine (EK)-containing peptide, or zwitterionic phosphatidyl serine moiety.

36. The method of claim 25, wherein the non-cationic lipid is selected from the group consisting of a dioleoylphosphatidylglycerol (DOPG), dipalmitoylphosphatidylglycerol (DPPG), dioleoylphosphatidylethanolamine (DOPE), palmitoyloleoyl-phosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl-phospho ethanolamine, 16-O-dimethyl-phosphoethanolamine, 18-1-trans-phosphoethanolamine, 1-stearoyl-2-oleoyl phosphatidyethanolamine (SOPE), and 1,2-dioleoyl-sn glycero-3-phophoethanolamine (transDOPE).

37. The method of claim 25, wherein component (ii) excludes a polyalkylene oxide segment.

38. The method of claim 25, wherein the non-cationic lipid in component (ii) is a phospholipid.

39. The method of claim 38, wherein the phospholipid is a phosphatidyl serine lipid.

40. The method of claim 25 wherein the cationic or ionizable lipid possesses a secondary, tertiary, or quaternary amino group.

41. The method of claim 25, wherein the cationic or ionizable lipid excludes a polyalkylene oxide segment.

42. The method of claim 25, wherein the lipid nanoparticle composition further comprises: (v) cholesterol or derivative thereof.

43. The method of claim 25, wherein the therapeutic substance is a nucleic acid molecule.

44. The method of claim 43, wherein the nucleic acid molecule is an RNA.

45. The method of claim 44, wherein the RNA is mRNA.

46. The method of claim 44, wherein the RNA is selected from the group consisting of message RNA (mRNA), small interfering RNA (siRNA), microRNA (miRNA), circularRNA (circRNA), long-noncoding RNA (lncRNA), antisense oligonucleotide (ASO), viral mRNA, CRISPR RNA, Cas nuclease mRNA, guide RNA, single-guide RNA, and combinations thereof.

47. The method of claim 25, wherein the therapeutic substance is a spike protein of a virus.

48. A lipid composition containing a lipid moiety attached to a secondary, tertiary, or quaternary amine group along with a functional group, wherein the functional group is negatively charged under physiological conditions.

49. A lipid composition comprising the lipid moiety of claim 15.

50. A lipid composition comprising the lipid moiety of claim 16.

51. A method for targeted delivery of a therapeutic agent to a secondary lymphoid organ (SLO) in a subject, the method comprising:
obtaining a lipid nanoparticle composition of any one of claims 1-24, the lipid nanoparticle composition further comprising a phosphoserine-containing lipid (PS); and
administering an effective amount of the lipid nanoparticle composition to the subject,
wherein the therapeutic agent is targeted to a secondary lymphoid organ (SLO) of the subject.

52. The method of claim 51, wherein the therapeutic agent comprises a nucleic acid molecule.

53. The method of claim 51, wherein the phosphoserine-containing lipid is selected from the group consisting of 1,2-dioleoyl-sn-glycero-3-phospho-L-serine (DOPS), naturally occurring PS-lipid, L-α-phosphatidylserine, and/or zwitterionic PS-lipid.

54. The method of claim 51, wherein the SLO is selected from spleen and lymph nodes.

55. The method of claim 51, wherein the targeted delivery of the therapeutic agent carries out cancer immunotherapy, autoimmune disease immunotherapy, vaccination and/or gene editing.

56. A method for targeted delivery of a therapeutic agent to macrophages in a subject, the method comprising:
obtaining a lipid nanoparticle composition of any one of claims 1-24, the lipid nanoparticle composition further comprising a phosphoserine-containing lipid (PS); and
administering an effective amount of the lipid nanoparticle composition to the subject,
wherein the therapeutic agent is targeted to macrophages of the subject.

57. The method of claim 56, wherein the therapeutic agent comprises a nucleic acid molecule.

58. The method of claim 56, wherein the phosphoserine-containing lipid is selected from the group consisting of 1,2-dioleoyl-sn-glycero-3-phospho-L-serine (DOPS), naturally occurring PS-lipid, L-α-phosphatidylserine, and/or zwitterionic PS-lipid.

59. The method of claim 56, wherein the therapeutic agent is targeted to macrophages of the subject thereby further targeting the therapeutic agent to the spleen and/or lymph nodes.

60. The method of claim 56, wherein the targeted delivery of the therapeutic agent carries out cancer immunotherapy, autoimmune disease immunotherapy, vaccination, and/or gene editing.

61. A method for targeted delivery of a therapeutic agent to a secondary lymphoid organ (SLO) in a subject, the method comprising:
obtaining a lipid nanoparticle composition, the lipid nanoparticle composition further comprising a phosphoserine-containing lipid (PS); and
administering an effective amount of the lipid nanoparticle composition to the subject,
wherein the therapeutic agent is targeted to a secondary lymphoid organ (SLO) of the subject.

62. A method for targeted delivery of a therapeutic agent to a secondary lymphoid organ (SLO) in a subject, the method comprising:
obtaining a lipid nanoparticle composition of any one of claims 48-50, the lipid nanoparticle composition further comprising a phosphoserine-containing lipid (PS); and
administering an effective amount of the lipid nanoparticle composition to the subject,
wherein the therapeutic agent is targeted to a secondary lymphoid organ (SLO) of the subject.

63. A method for targeted delivery of a therapeutic agent to macrophages in a subject, the method comprising:
obtaining a lipid nanoparticle composition, the lipid nanoparticle composition further comprising a phosphoserine-containing lipid (PS); and
administering an effective amount of the lipid nanoparticle composition to the subject,
wherein the therapeutic agent is targeted to macrophages of the subject.

64. A method for targeted delivery of a therapeutic agent to macrophages in a subject, the method comprising:
obtaining a lipid nanoparticle composition of any one of claims 48-50, the lipid nanoparticle composition further comprising a phosphoserine-containing lipid (PS); and
administering an effective amount of the lipid nanoparticle composition to the subject,
wherein the therapeutic agent is targeted to macrophages of the subject.
